(19) United States
(12) Patent Application Publication
(10) Pub. No.: US 2023/0270692 A1
(43) Pub. Date: Aug. 31, 2023

(54) COMPOUNDS FOR CHRONIC DISORDERS

(71) Applicant: Pillai Universal LLC, Carson City, NV (US)

(72) Inventors: Baskaran PILLAI, Pittsburgh, PA (US); Murugan Girija DINESH, Ambattur, Chennai (IN)

(21) Appl. No.: 17/792,020

(22) PCT Filed: Jan. 11, 2021
(86) PCT No.: PCT/US2021/012959

§ 371 Date: Jul. 11, 2022

Related U.S. Application Data
(60) Provisional application No. 63/005,730, filed on Apr. 6, 2020.

(30) Foreign Application Priority Data
Jan. 10, 2020 (IN) 202041001163

(51) Int. Cl. A61K 31/12; A61K 31/4706; A61P 31/14; A61P 35/00; A61K 31/704; A61K 31/7068; A61K 31/513; A61K 33/243; A61K 31/454; A61K 31/4745; A61K 31/475; A61K 31/05; A61K 31/15; C07C 49/255; C07C 49/258; C07C 43/253; C07C 251/16

(52) U.S. Cl. CPC A61K 31/12; A61K 31/4706; A61P 31/14; A61P 35/00; A61K 31/704; A61K 31/7068; A61K 31/513; A61K 33/243; A61K 31/454; A61K 31/4745; A61K 31/475; A61K 31/05; A61K 31/15; C07C 49/255; C07C 49/258; C07C 43/253; C07C 251/16

(57) ABSTRACT

The present disclosure provides compounds and compositions that are useful in treating chronic disorders, including cancer and viral diseases.

### CROSS REFERENCE TO RELATED APPLICATIONS

This application is a U.S. National Stage Application under 35 U.S.C. 371 International Application No. PCT/US2021/012959, filed Jan. 11, 2021, which claims the benefit of and priority to U.S. Provisional Application No. 63/005,730, filed Apr. 6, 2020, and Indian Application No. 202041001163, filed Jan. 10, 2020, each of which is herein incorporated by reference in its entirety

### BACKGROUND OF THE INVENTION

A Chronic condition is a human health condition or disease that is persistent or otherwise long-lasting in its effects or a disease that comes with time. The term chronic is often applied when the course of the disease lasts for more than three months. Common chronic diseases include arthritis, asthma, cancer, chronic obstructive pulmonary disease, diabetes and some viral diseases such as hepatitis C and acquired immunodeficiency syndrome.

Cancer, also called malignancy, is an abnormal growth of cells. Cancer develops when the body's normal control mechanism stops working. Old cells do not die and instead grow out of control, forming new, abnormal cells. These extra cells may form a mass of tissue, called a tumor. Some cancers, such as leukemia, do not form tumors.

There are more than 100 types of cancer, including breast cancer, skin cancer, lung cancer, colon cancer, prostate cancer, and lymphoma. Symptoms vary depending on the type.

Treatment options depend on the type of cancer and its stage. The treatment is aimed to kill as many cancerous cells while reducing damage to normal cells nearby.

The three main treatments are:
Surgery: directly removing the tumor
Chemotherapy: using chemicals to kill cancer cells
Radiation therapy: using X-rays to kill cancer cells

The major unresolved problems with metastatic cancer are recurrence after receiving an objective response to chemotherapy, drug-induced side effects of first-line chemotherapy and delayed response to second line of treatment. Unfortunately, very few options are available as a third-line treatment. Thus, there is growing need to find new chemopreventive agents that may be effective in prevention and/or management of chronic conditions, such as cancer.

Natural products such as flavonoids can be useful for prevention or treatment of chronic disorders. However, because of reduced solubility and bio-availability, the natural products are not efficient alternative to current therapeutic agents. Thus, there exists a need in the state of art to develop novel agents which provide increased bioavailability, solubility and tissue distribution abilities for targeted delivery. Such agents may be active against a variety of chronic conditions and, in some embodiments, may exhibit anticancer or antiviral activity.

### BRIEF DESCRIPTION OF DRAWINGS

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 depicts the spectrum of compound I
a. Mass Spectrum
b. IR Spectrum
c. ¹H NMR Spectrum
d. ¹³C NMR Spectrum

FIG. 2 depicts the spectrum of compound II
a. Mass Spectrum
b. IR Spectrum
c. ¹H NMR Spectrum
d. ¹³C NMR Spectrum

FIG. 3 depicts the spectrum of compound III
a. Mass Spectrum
b. IR Spectrum
c. ¹H NMR Spectrum
d. ¹³C NMR Spectrum

FIG. 4 depicts the spectrum of compound IV
a. Mass Spectrum
b. IR Spectrum
c. ¹H NMR Spectrum
d. ¹³C NMR Spectrum

FIG. 5 depicts the spectrum of compound V
a. Mass Spectrum
b. IR Spectrum
c. ¹H NMR Spectrum
d. ¹³C NMR Spectrum

FIG. 6 depicts the spectrum of compound VI
a. Mass Spectrum
b. IR Spectrum
c. ¹H NMR Spectrum
d. ¹³C NMR Spectrum

FIG. 7 depicts the spectrum of compound VII
a. Mass Spectrum
b. IR Spectrum
c. ¹H NMR Spectrum
d. ¹³C NMR Spectrum

FIG. 8 depicts the spectrum of compound VIII
a. Mass Spectrum
b. IR Spectrum
c. ¹H NMR Spectrum
d. ¹³C NMR Spectrum

FIG. 9 depicts the spectrum of compound IX
a. Mass Spectrum
b. IR Spectrum
c. ¹H NMR Spectrum
d. ¹³C NMR Spectrum

FIG. 10A depicts the anticancer activity of Compound Ion different cell lines.
A: Effect of molecules on different cancer cell lines;
B: Effect of molecules on normal cell lines;
C: Comparison with other standard drugs:
Abbreviation
MCF-7 (Human breast cancer cells);
MDAMB 231 (Triple-negative Breast Cancer Cell Line);
PANC-1 (Human pancreatic cancer cell lines);
HT-29 (Human Colon cancer cell lines);
T-ALL (T-cell acute lymphoblastic leukemia);
HDF (Human Dermal Fibroblast);
AC-16 (Human Cardiomyocytes cells);
HBMSC (Human Bone marrow Mesenchymal stem cells);
MCF-12 A (Human Normal breast cells);
CRL2989 (Human normal pancreatic cells);
NCM 60 (Normal Colon Epithelial cell lines)

FIG. 10B depicts apoptosis induction and mitochondrial membrane potential for control vs. Compound I.

FIG. 11 depicts a series of graphs used to elucidate the mechanism of action of compounds of the present disclosure (e.g., Compound I) that inhibit histone deacetylase (HDAC) activity.

FIG. 12 depicts a series of graphs showing that compound I induced Neuronal cell type formation
a. Human stem cells—Neuronal differentiation of molecules
b. Tumor Gene Expression analysis of molecules

FIG. 13 depicts the inhibition of cancer stem cells markers by Compound I.

FIG. 14 depicts cancer cell dynamics and treatment for conventional drugs vs. the compounds of the present disclosure.

FIG. 15 depicts breakthroughs achieved by the compounds of the present disclosure.

FIG. 16 depicts cancer stem cells and receptors.

FIG. 17 depicts aberrant signal transduction pathways in cancer stem cells and shows cancer stem cells as targets.

FIG. 18 depicts the genes targeted by the compounds of the disclosure for various types of cancers.

FIGS. 19A-19B depicts the cytotoxicity of anticancer agents assessed by measuring IC50 against a panel of normal cell lines treated without Compound 1 (FIG. 19A) and in combination with Compound I (FIG. 19B).

FIG. 20A-20C depict in vivo studies in a stage IV metathesis tumor model comparing convention drugs to Compound I of the present disclosure.

FIGS. 21A-21D provide a PANC-1 mouse xenograft model evaluating the efficacy of Compound I compared to gemcitabine.

FIG. 22 depicts the steps in the Coronavirus replication pathway targeted by the compounds of the disclosure.

FIG. 23 depicts how binding of an ACE-2 inhibitor disrupts the interaction between virus and receptor.

FIG. 24A depicts the molecular binding sites of Nsp15.

FIG. 24B depicts a model showing binding of a small molecule with Nsp15.

FIG. 24C depicts a model showing the binding site residues that are proposed to interact with the compounds of the disclosure.

FIG. 25A depicts normal Vero cells and Vero cells infected with SARS-CoV-2 at MOI of 0.1 in the treatment of different doses of the indicated antivirals for 48 h. The viral yield in the cell supernatant was then quantified by qRT-PCR.

FIG. 25B depicts graphs representing the mean % inhibition of virus yield and cytotoxicity of the drugs (e.g., Compound I), respectively. The experiments were done in triplicate.

FIG. 25C depicts immunofluorescence microscopy of virus infection upon treatment of Compound I. At 48 h post-infection, the infected cells were fixed, and then probed with rabbit sera against the nucleoprotein (NP) of SARS-related CoV as the primary antibody and Alexa 488-labeled goat anti-rabbit IgG as the secondary antibody, respectively. The nuclei were stained with Hoechst dye. Bars, 20 μm.

FIG. 25D depicts Western blot analysis of nucleoprotein (NP) expression at 24 h post infection (p.i.) in cells infected with SARS-CoV-2 at MOI of 0.1.

FIG. 25E depicts a graph representing the expression of nucleoprotein (NP) normalized to GAPDH. Virus yield in the infected cell supernatants was quantified by qRT-PCR. Experiments were done in triplicate.

FIGS. 25F-25H depict graphs showing a reduction in viral RNA was found in both supernatant and cell pellets from samples treated with 11 μM of Compound I.

FIG. 26A depicts a cell viability assay for calu-3 cells treated with different concentrations of Compound I.

FIG. 26B depicts percent plaque reduction assay in calu-3 cells treated with different concentrations of Compound I.

FIG. 27A depicts the H&E staining analysis of liver and kidney samples from a tissue distribution study in nude mice administered a single dose of 10 mg/kg by weight of Compound I.

FIG. 27B depicts the results of tissue distribution studies from 0.5 h to 96 h in the heart, lungs, muscles, spleen, tibia, and femur of nude mice administered a single dose of 10 mg/kg by weight of Compound I.

FIGS. 28A-28E are graphs showing the effect on the organ weight (FIG. 28A-28B), liver weight (FIG. 28C), and body weight (FIG. 28D-28E) of male and female Wistar rats upon oral administration of various amounts of Compound I.

FIGS. 28F-28I are graphs showing the effect on haematological parameters (urea, creatinine, etc.) when male and female Wistar rats are orally administered various amounts of Compound I.

FIGS. 28J-28K are graphs showing the effect on electrolytes when male and female Wistar rats are orally administered various amounts of Compound I.

FIGS. 28L-280 are graphs showing the effect on liver function of male and female Wistar rats upon oral administration of various amounts of Compound I.

FIGS. 28P-28Q are graphs showing the effect on kidney function of male and female mice upon oral administration of various amounts of Compound I.

FIGS. 29A-29B depict histopathological studies from the analysis of the organ samples of liver, kidney, skeletal muscle, heart, and spleen from both male (FIG. 29A) and female rats (FIG. 29B). Tissue samples were collected on the final day of the treatment period.

### SUMMARY OF THE INVENTION

The present disclosure is related to compounds with anticancer and antiviral activity. The compounds may be used for treating subjects with chronic disorder. The compounds have a structure in of Formula 1A

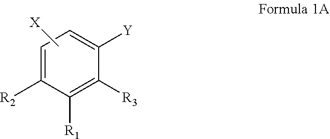

or any derivative thereof, pharmaceutically acceptable salt thereof, or combination thereof

in which:
R₁ is H, OH, or alkoxy;
R₂ is alkoxy, or OH;
R₃ is alkoxy or OH;
X is C₁-C₁₅ alkyl, C₂-C₁₅ alkenyl, C₂-C₁₅ alkynyl, or aralkyl chain, each of which is independently substituted with at least one alkoxy, OH, ═NH, or oxo, group;
Y is H or alkyl; and
X is in ortho position to R₂, or para position to R₁.

### DETAILED DESCRIPTION OF THE INVENTION

### Definitions

“Alkyl” or “alkyl group” refers to a fully saturated, straight or branched hydrocarbon chain having from one to fifteen carbon atoms, and which is attached to the rest of the molecule by a single bond. Unless stated otherwise specifically in the specification, an alkyl group can be optionally substituted.

“Alkenyl” or “alkenyl group” refers to a straight or branched hydrocarbon chain having from two to fifteen carbon atoms, and having one or more carbon-carbon double bonds. Each alkenyl group is attached to the rest of the molecule by a single bond. Unless stated otherwise specifically in the specification, an alkylene chain can be optionally substituted.

“Alkenylene” or “alkenylene chain” refers to a straight or branched divalent hydrocarbon chain radical, having from two to twelve carbon atoms, and having one or more carbon-carbon double bonds. The alkenylene chain is attached to the rest of the molecule through a single bond and to the radical group through a single bond. Unless stated otherwise specifically in the specification, an alkenylene chain can be optionally substituted.

“Alkoxy” refers to a group of the formula —ORₐ where Rₐ is an alkyl, alkenyl or alkynyl as defined above containing one to twelve carbon atoms. Unless stated otherwise specifically in the specification, an alkoxy group can be optionally substituted.

Aralkyl” or “arylalkyl” refers to a group of the formula —R_{b}—R_{c}, where R_{b} is an alkylene or alkenylene group as defined above and R_{c} is one or more aryls as defined above, for example, benzyl, diphenylmethyl and the like. Unless stated otherwise specifically in the specification, an aralkyl group can be optionally substituted.

“Oxo” refers to the following group with a double bound to an oxygen atom.

The term “substituted” used herein means any of the above groups, wherein at least one hydrogen atom is replaced by a bond to a non-hydrogen atoms, including but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

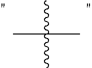

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

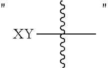

indicates that the chemical entity “XY” is bonded to another chemical entity via the point of attachment bond. Furthermore, the specific point of attachment to the non-depicted chemical entity can be specified by inference. For example, the compound CH₃—R₃, wherein R₃ is H or

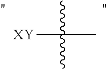

infers that when R₃ is “XY”, the point of attachment bond is the same bond as the bond by which R₃ is depicted as being bonded to CH₃.

“Pharmaceutically acceptable salts” include, when appropriate, pharmaceutically acceptable base addition salts and acid addition salts, for example, metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Other examples of acid addition salts include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts. When the compounds contain an acidic side chain, suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

The term “therapeutically effective” applied to dose or amount refers to that quantity of a compound or pharmaceutical formulation that is sufficient to result in a desired clinical benefit after administration to a patient in need thereof.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally the present invention discloses novel therapeutic compounds for treating chronic disorders which includes cancer.

In some embodiments, the present invention discloses a compound having a structure of Formula 1A

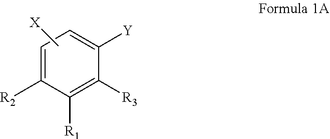

or any derivative thereof, pharmaceutically acceptable salt thereof, or combination thereof

in which:
R₁ is H, OH, or alkoxy;
R₂ is alkoxy, or OH;
R₃ is alkoxy or OH;
X is C₁-C₁₅ alkyl, C₂-C₁₅ alkenyl, or aralkyl chain, each of which is independently substituted with at least one alkoxy, OH, ═NH, or oxo, group;
Y is H or alkyl; and
X is in ortho position to R₂, or para position to R₁.

In some embodiments of the compound of Formula 1A,
R₁ is H.
R₂ is —OH;
R₃ is C₁-C₃alkoxyl; and
X is C₄-C₈ alkenyl substituted with 2 oxo groups
Y is C₁-C₃ alkyl.

In some embodiments, the compound of Formula 1A has a structure of Formula 1

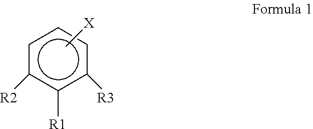

or any derivative thereof, pharmaceutically acceptable salt thereof, or combination thereof

in which:
R₁ is H, alkoxy, or OH;
R₂ is alkoxy or OH;
R₃ is alkoxy or OH;
X is C₁-C₁₅ alkyl, C₂-C₁₅ alkenyl, or aralkyl, chain, each of which is independently substituted with at least one alkoxy, OH, ═NH, or oxo group.

In some embodiments of Formula 1, X is
(i) C₆-C₁₀ alkenyl, substituted with four substituents independently selected from the group consisting of oxo, —OH, and C₁-C₃ alkoxy;
(ii) C₈-C₁₂ alkenyl substituted with oxo and C₁-C₃ alkoxy;
(iii) C₄-C₈ alkenyl substituted with 2 oxo groups;
(iv) C₂-C₆ alkenyl substituted with oxo and C₁-C₃ alkoxy;
(v) C₁-C₆ alkyl substituted with ═NH;
(vi) C₂-C₃ alkenyl substituted with —OH;
(vii) C₈-C₁₂ alkenyl substituted with oxo and 2 C₁-C₃ alkoxy groups; or
(viii) aralkyl comprising a C₆-C₈ alkyl and a C₆ aryl, wherein the alkyl is substituted with oxo and the aryl is substituted with two alkoxy groups.

In some embodiments of Formula 1,
R₁ is H;
R₂ is C₁-C₃ alkoxy;
R₃ is C₁-C₃ alkoxy; and
X is C₆-C₁₀ alkenyl, substituted with oxo, —OH, and 2 C₁-C₃ alkoxy groups;

In some embodiments of Formula 1,
R₁ is H;
R₂ is C₁-C₃ alkoxy;
R₃ is C₁-C₃ alkoxy; and
X is C₈-C₁₂ alkenyl substituted with oxo and C₁-C₃ alkoxy;

In some embodiments of Formula 1,
R₁ is OH;
R₂ is C₁-C₃ alkoxy;
R₃ is C₁-C₃ alkoxy; and
X is C₄-C₈ alkenyl substituted with two oxo groups;

In some embodiments of Formula 1,
R₁ is C₁-C₃ alkoxy;
R₂ is OH;
R₃ is C₁-C₃ alkoxy; and
X is C₄-C₈ alkenyl substituted with 2 oxo groups;

In some embodiments of Formula 1,
R₁ is H;
R₂ is OH;
R₃ is OH; and
X is C₁-C₆ alkyl substituted with ═NH;

In some embodiments of Formula 1,
R₁ is OH;
R₂ is C₁-C₃ alkoxy;
R₃ is C₁-C₃ alkoxy; and
X is C₂-C₃ alkenyl substituted with OH.

In some embodiments of Formula 1,
R₁ is H;
R₂ is C₁-C₃ alkoxy;
R₃ is C₁-C₃ alkoxy; and
X is C₈-C₁₂ alkenyl substituted with oxo and two C₁-C₃ alkoxy groups

In some embodiments of Formula 1,
R₁ is OH;
R₂ is C₁-C₃ alkoxy;
R₃ is C₁-C₃ alkoxy; and
X is aralkyl comprising a C₆-C₈ alkyl and a C₆ aryl, wherein the alkyl is substituted with oxo and the aryl is substituted with two C₁-C₃ alkoxy groups.

In some embodiments, the compound of Formula 1A has a structure of Formula 2

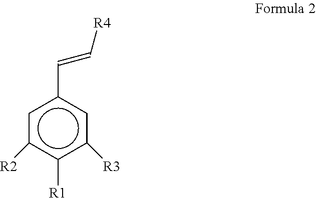

or any derivative thereof, pharmaceutically acceptable salt thereof, or combination thereof

in which
R₁ is H or OH;
R₂ is alkoxy or OH;
R₃ is alkoxy or OH; and
R₄ is C₁-C₁₅ alkyl, C₂-C₁₅ alkenyl, aralkyl, each of which is substituted with at least one alkoxy, —OH, or oxo.

In some embodiments of Formula 2, R₄ is:

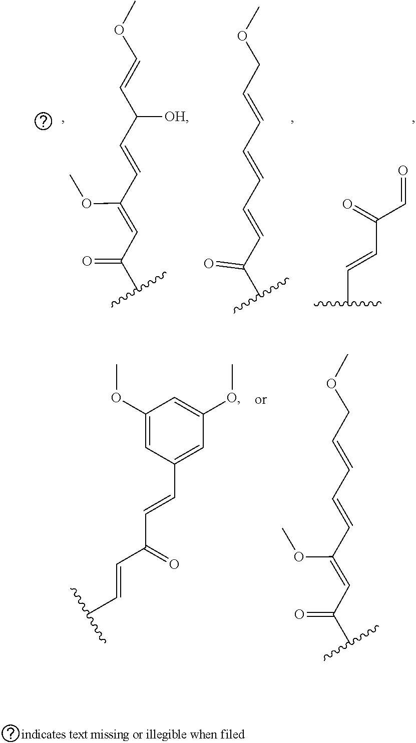 [SMILES: *C(=O)/C=C(/C=C/C(O)/C=C/OC)OC ; *C(=O)/C=C/C=C/C=C/COC ; */C=C/C(=O)C=O ; */C=C/C(=O)/C=C/c1cc(OC)cc(OC)c1 ; *C(=O)/C=C(/C=C/C=C/COC)OC]

In some embodiments of Formula 2,
R₁ is H;
R₂ is OCH₃;
R₃ is OCH₃; and
R₄ is

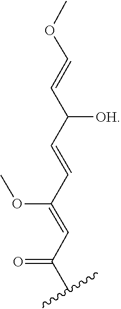 [SMILES: *C(=O)/C=C(/C=C/C(O)/C=C/OC)OC]

In some embodiments of Formula 2,
R₁ is H;
R₂ is OCH₃;
R₃ is OCH₃; and
R₄ is.

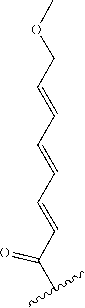 [SMILES: *C(=O)/C=C/C=C/C=C/COC]

In some embodiments of Formula 2,
R₁ is OH;
R₂ is OCH₃;
R₃ is OCH₃; and

R₄ is

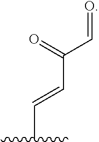 [SMILES: */C=C/C(=O)C=O]

In some embodiments of Formula 2,
R₁ is OH;
R₂ is OCH₃;
R₃ is OCH₃; and
R₄ is

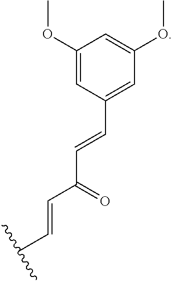 [SMILES: */C=C/C(=O)/C=C/c1cc(OC)cc(OC)c1]

In some embodiments of Formula 2,
R₁ is H;
R₂ is OCH₃;
R₃ is OCH₃; and
R₄ is

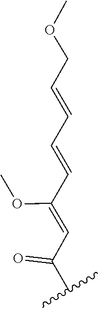 [SMILES: *C(=O)/C=C(/C=C/C=C/COC)OC]

In some embodiments, Formula 1A has a structure of Formula 3

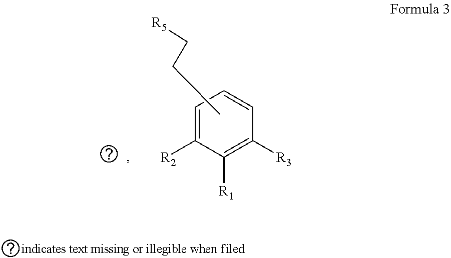

or any derivative thereof, pharmaceutically acceptable salt thereof, or combination thereof

in which:
R₁ is H, alkoxy, or OH;
R₂ is alkoxy or OH;
R₃ is alkoxy or OH; and
R₅ is C₁-C₁₂ alkyl or C₂-C₁₂ alkenyl, which is independently substituted with 1, or 2, substituents selected from the group consisting of: ═NH or oxo.

In some embodiments of Formula 3,
R₁ is H;
R₂ is OH;
R₃ is OH; and
R₅ is ═NH.
In some embodiments, R₅ is in the ortho position to R₂.

In some embodiments of Formula 3,
R₁ is —OC₂H₅;
R₂ is OH;
R₃ is —OCH₃; and
R₅ is

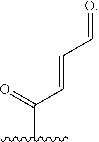 [SMILES: *C(=O)/C=C/C=O]

In some embodiments of Formula 3, R₅ is in the para position to R₁.

In some embodiments, the compound of Formula 1A has a structure of Formula 4

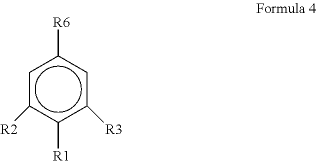

or any derivative thereof, pharmaceutically acceptable salt thereof, or combination thereof

in which:
R₁ is OH;
R₂ is C₁-C₃ alkoxy;
R₃ is C₁-C₃ alkoxy; and
R₆ is C(CH₂)OH.

In some embodiments of Formula 4,
R₁ is OH;
R₂ is OCH₃;
R₃ is OCH₃; and
R₆ is C(CH₂)OH.

In some embodiments, the present invention provides a pharmaceutical composition, comprising one of the compounds described above and a pharmaceutically acceptable carrier.

In some embodiments of the present disclosure, the pharmaceutical acceptable carrier comprises acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxpropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate,

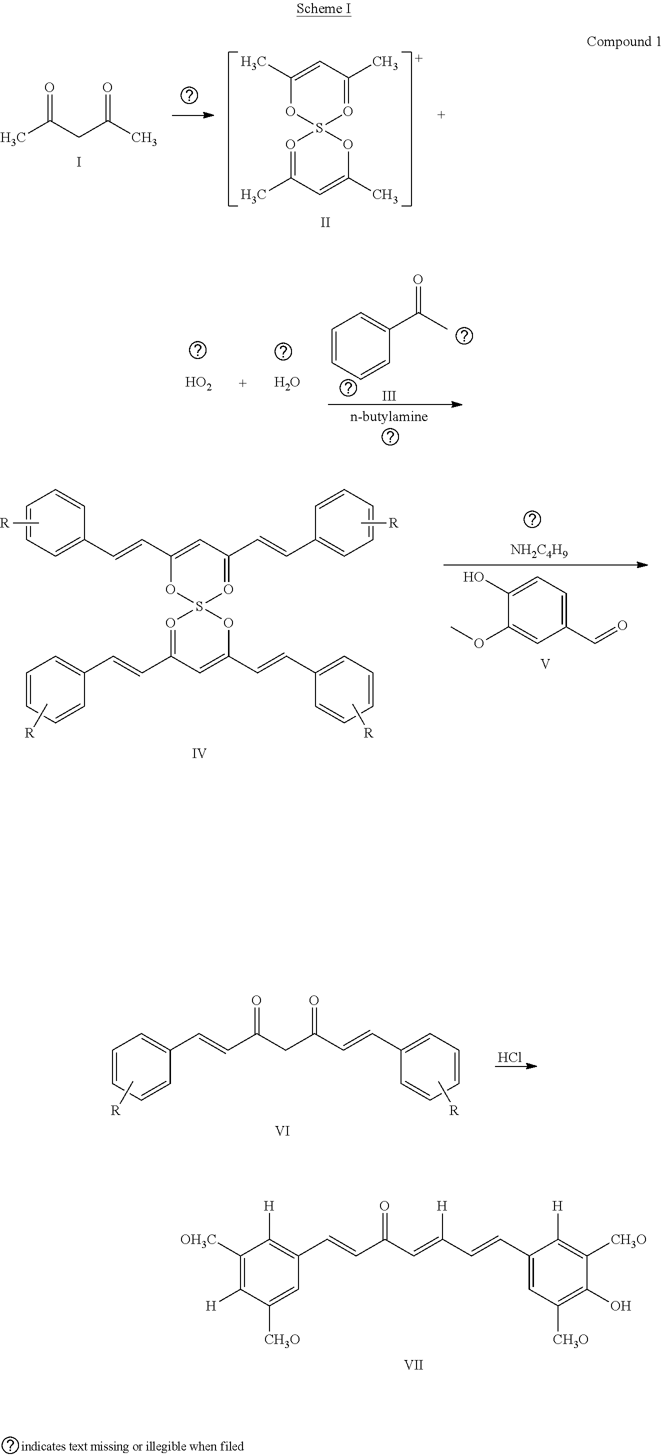

sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or a combinations thereof.

The chemical structures for any of Formulae 1A-4 can be prepared according to the example synthetic route disclosed herein.

In one embodiment, example Compound I of Formula 1A can be prepared by the Scheme I provided below.

Chemical synthesis: In dry three-necked flask acetylacetone (5 mmol, 0.51 ml) and boron oxide (3.5 mmol, 0.244 g) was dissolved in absolute ethyl acetate and stirred for 30 min at 40° C. Then the corresponding aldehyde (10 mmol) and tributyl borate (10 mmol, 2.4 ml) was added and stirred for another 30 min. n-Butylamine (7.5 mmol) was dissolved in dry ethyl acetate and then added over a period of 15 min. The mixture was heated to 40° C. for 24 h. Then 5 ml of HCl (10%) were added and heated to 60° C. for an additional hour. The aqueous phase was extracted with ethyl acetate several times, the organic layers were dried over Na₂SO₄ and the solvent distilled off. An insoluble precipitate (part of the product) was filtered off and recrystallized with the residue from various solvents. Purification was carried out with column chromatography (eluent toluene/ethyl acetate 8:2) and crystallization from ethanol (80%).

The synthesized compounds of Formula 1A-4 were next subject to molecular characterization to ascertain the structure of the compounds.

Characterization:

The purity of synthesized products verified by the melting point, Thin Layer Chromatography, HPLC, IR, Mass Spectroscopy and NMR analysis. From FIG. 1-9, the structure of the synthesized compound of Formula 1A-4 is elucidated as Compound I to IX as follows in Table 1.

TABLE 1

| Compound | Structure |
|---|---|
| I | 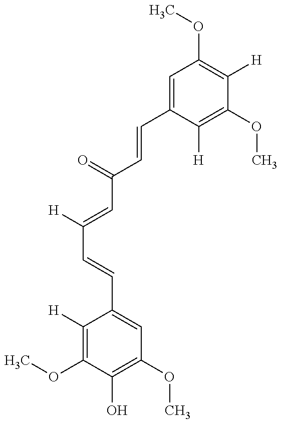 [SMILES: COc1cc(/C=C/C(=O)/C=C/C=C/c2cc(OC)c(O)c(OC)c2)cc(OC)c1] |
| II | 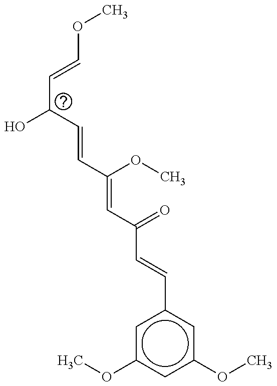 |
| III | 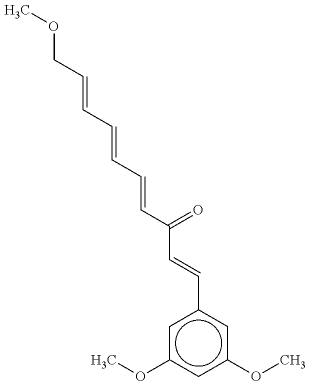 |
| IV | 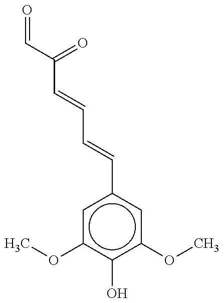 |
| V | 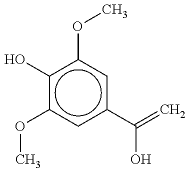 |
| VI | 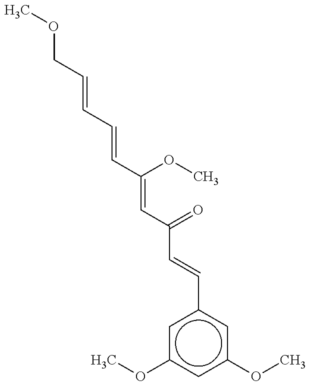 |
| VII | 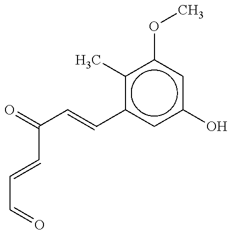 |
| VIII | 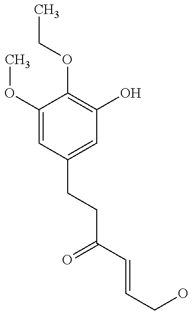 [SMILES: CCOc1c(O)cc(CCC(=O)/C=C/CO)cc1OC] |
| IX | 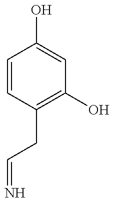 [SMILES: N=CCc1ccc(O)cc1O] |

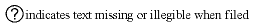

In some embodiments, the compounds of Formula 1A-4 and the compounds I-IX may encompass a cis-isomer, trans-isomer, or both cis- and trans-isomers. In some embodiments, the compounds of Formula 1A-4 and the compounds I-IX may be a mixture of cis- and trans-isomers. In some embodiments, the compounds of Formula 1A-4 and the compounds I-IX may be cis-isomers (i.e., Z-isomers). In some embodiments, the compounds of Formula 1A-4 and the compounds I-IX may be trans-isomers (i.e., E-isomers).

In some embodiments, the compound of Formula 1A-4 and the compounds I-IX may encompass either R or S stereoisomers and be a mixture of stereoisomers (e.g., a mixture of diastereomers. In some embodiments, the compound of Formula 1A-4 may be a racemic mixture or enantiopure.

In some embodiments, the compound of Formula 1A-4 and the compounds I-IX are enantiopure, (e.g., comprise either R or S enantiomers), diastereomerically pure, or comprise a mixture of stereoisomers (e.g., a racemic mixture or a mixture of diastereomers). In some embodiments, the compound of Formula 1A-4 is a racemic mixture. In some embodiments, the compound of Formula 1A-4 is enantiopure. In some embodiments, the compound of Formula 1A-4 is diastereomerically pure.

In some embodiments, an enantiopure compound is a compound that has an enantiomeric excess (ee) of greater than about 75%, greater than about 80%, greater than about 85%, greater than about 90%, greater than about 95%, or greater than about 99%.

In some embodiments, a diastereomerically pure compound is compound that has a diastereomeric excess (de) of greater than about 75%, greater than about 80%, greater than about 85%, greater than about 90%, greater than about 95%, or greater than about 99%.

The compounds of the present inventions can be used to perform or provide any of the biological functions, described herein.

Pharmaceutical Compositions

The present disclosure also includes pharmaceutical compositions comprising a therapeutically effective amount of one or more compounds disclosed herein. In some embodiments, pharmaceutical compositions comprise a therapeutically effective amount of one or more compounds of Formula 1A, 2, 3, and/or 4, or pharmaceutically acceptable salts thereof. In other embodiments, pharmaceutical compositions comprise a therapeutically effective amount of one or more compounds selected from Table 1, or pharmaceutically acceptable salts thereof.

In various aspects, the amount of compounds of Formula 1A-4 (including compounds in Table 1), or a pharmaceutically acceptable salt thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be once administered daily or more frequently depending upon a number of factors, including the overall health of a patient, and the formulation and route of administration of the selected compound(s).

The compounds or pharmaceutical compositions of the present disclosure may be manufactured and/or administered in single or multiple unit dose forms.

In some embodiments, the compounds of the present disclosure (compounds of Formula 1-4, and Table 1) are administered to a patient with a chronic condition. In the context of some embodiments of the present invention, the term “chronic disorder” refers, but not limited to acute lymphoblastic, acute lymphoblastic leukemia, acute lymphocytic leukemia, acute myelogenous leukemia, acute myeloid leukemia, adrenocortical carcinoma, AIDS-related lymphoma, anal cancer, appendix cancer, basal-cell carcinoma, bladder cancer, brain cancer, brainstem glioma, breast cancer, bronchial adenomas/carcinoids, Burkitt's lymphoma, carcinoid tumor, cerebellar or cerebral astrocytoma, cervical cancer, cholangiocarcinoma, chondrosarcoma, chronic lymphocytic or chronic lymphocytic leukemia, chronic myelogenous or chronic myeloid leukemia, chronic myeloproliferative disorders, colon cancer, cutaneous T-cell lymphoma, desmoplastic small round cell tumor, endometrial uterine cancer, ependymoma, esophageal cancer, Ewing's sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal tumor (GIST), gestational trophoblastic tumor, glioma of the brain stem, hairy cell leukemia, head and neck cancer, heart cancer, hepatocellular (liver) cancer, Hodgkin lymphoma, hypopharyngeal cancer, hypothalamic and visual pathway glioma, intraocular melanoma, Islet cell carcinoma, Kaposi sarcoma, laryngeal cancer, leukaemia, lip and oral cavity cancer, liposarcoma, lymphoma, male breast cancer, malignant mesothelioma, medulloblastoma, melanoma, Merkel cell skin carcinoma, mesothelioma, metastatic squamous neck cancer, mouth cancer, multiple endocrine neoplasia syndrome, multiple myeloma, multiple myeloma/plasma cell neoplasm, mycosis fungoides, myelodysplastic/myeloproliferative diseases, nasal cavity and paranasal sinus cancer, nasopharyngeal carcinoma, neuroblastoma, non-Hodgkin lymphoma, non-melanoma skin cancer, non-small cell lung cancer, oligodendroglioma, oral cancer, oropharyngeal cancer, osteosarcoma and malignant fibrous histiocytoma, ovarian cancer, ovarian germ cell tumor, ovarian epithelial cancer (surface epithelial-stromal tumor), ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonary blastoma, primary carcinoma, primary central nervous system lymphoma, primary liver cancer, prostate cancer, rectal cancer, renal cell carcinoma, renal pelvis and ureter carcinoma, retinoblastoma, rhabdomyosarcoma, salivary gland cancer, Sezary syndrome, small cell lung cancer, small intestine cancer, soft tissue sarcoma, squamous cell carcinoma, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine sarcoma, vaginal cancer, visual pathway and hypothalamic glioma, vulvar cancer, Waldenstrom macroglobulinemia, Wilms tumor, Parkinson's disease and Parkinsonian disorders, Huntington's disease, Alzheimer's disease, multiple sclerosis, amyotrophic lateral sclerosis, Shy-Drager syndrome, progressive supranuclear palsy, Lewy body disease, spinal ischemia, spinal cord injuries, ischemic stroke, cerebral infarction, spinal cord injury, and cancer-related brain and spinal cord injury, multi-infarct dementia, geriatric dementia, other cognitive impairments, depression, Onychomycosis (fungal infection of the nail, Gingivitis and Periodontal Disease (Gum Disease) obesity and diabetics. The compounds disclosed herein are also good candidates for Severe Acute Respiratory Syndrome (SARS) and Coronavirus disease 2019 (COVID-19), as well as different cancers with KRAS oncogene mutation.

In some embodiments, the chronic condition is cancer. In some embodiments, the cancer is colon cancer, prostate cancer, breast cancer, or leukemia. In some embodiments, the cancer is a stage 4 cancer. In some embodiments, the colon cancer, prostate cancer, breast cancer, or leukemia is stage 4. In some embodiments, the chronic condition is KRAS oncogene mutation in various cancers.

In some embodiments, the chronic condition is a viral infection such as SARS or COVID-19.

In certain embodiments, the methods, compounds, and compositions described herein are administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing.

Alternatively, or in combination with the aforesaid combinations, the methods and compositions described herein can be administered in combination with one or more of: a vaccine, e.g., a therapeutic cancer vaccine; or other forms of cellular immunotherapy.

In another embodiment, the methods, compounds, and compositions described herein are used in combination with one, two or all of oxaliplatin, leucovorin or 5-FU (e.g., a FOLFOX co-treatment). Alternatively or in combination, the combination further includes a VEGF inhibitor (e.g., a VEGF inhibitor as disclosed herein).

Non-limiting examples of additional therapeutic agents which can be combined with the methods disclosed herein include: taxol, imatinif, doxorubicin, paclitaxel, fluorouracil (5-FU), and vinblastin.

In some embodiments, the methods and compositions described herein can be administered in combination with one or more antiviral agents.

Non-limiting examples of additional therapeutics (e.g., antiviral agents) which can be combined with the methods disclosed herein include: remdesivir, lopinavir/ritonavir, favilavir, chloroquine, hydroxychlorquine, azithromycin, or combinations thereof.

### Numbered Embodiments

1. A compound of Formula 1A,

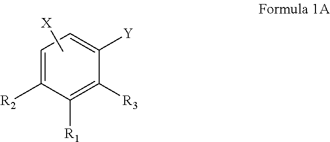

or a pharmaceutically acceptable salt thereof,

in which:
R₁ is H, OH, or alkoxy;
R₂ is alkoxy, or OH;
R₃ is alkoxy or OH;
X is C₁-C₁₅ alkyl, C₂-C₁₅ alkenyl, C₂-C₁₅ alkynyl, or aralkyl, chain, each of which is independently substituted with at least one alkoxy, OH, ═NH, or oxo, group;
Y is H or alkyl; and
X is in ortho position to R₂, or para position to R₁.

2. The compound of embodiment 1, in which:
R₂ is C₁-C₃ alkoxy.

3. The compound of embodiment 1 or 2, in which:
R₃ is C₁-C₃ alkoxy.

4. The compound of embodiments 1-3, in which:
X is C₁-C₁₅ alkyl or C₂-C₁₅ alkenyl each of which is independently substituted with 1, 2, 3, or 4 substituents selected from the group consisting of: alkoxy, OH, ═NH, or, oxo group

5. The compound of embodiments 1-4, in which:
R₁ is H or OH.

6. The compound of embodiments 1-5, in which:
Y is H.

7 The compound of embodiments 1-5, in which:
R₁ is H;
R₂ is —OH;
R₃ is C₁-C₃alkoxyl;
X is C₄-C₈ alkenyl substituted with two oxo groups; and
Y is C₁-C₃ alkyl.

8. A compound of embodiment 1, having a structure of Formula 1

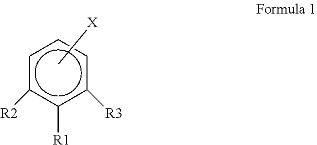

or a pharmaceutically acceptable salt thereof,

in which:
R₁ is H, alkoxy, or OH;
R₂ is alkoxy or OH;
R₃ is alkoxy or OH; and
X is C₁-C₁₅ alkyl, C₂-C₁₅ alkenyl, C₂-C₁₅ alkynyl, or aralkyl, chain, each of which is independently substituted with at least one alkoxy, OH, ═NH, or oxo group;

9. The compound of embodiment 8, in which:
R₁ is H or OH;
R₂ is C₁-C₃alkoxy or OH;
R₃ is C₁-C₃alkoxy or OH; and
X is C₁-C₁₂ alkyl, C₂-C₁₂ alkenyl, or aralkyl each of which is independently substituted with 1, 2, 3, or 4 substituents independently selected from the groups consisting of alkoxy, OH, ═NH and oxo group

10. The compound of embodiment 8 or 9, in which X is:
(i) C₆-C₁₀ alkenyl, substituted with four substituents independently selected from the group consisting of oxo, —OH, and C₁-C₃ alkoxy;
(ii) C₈-C₁₂ alkenyl substituted with oxo and C₁-C₃ alkoxy;
(iii) C₄-C₈ alkenyl substituted with 2 oxo groups;
(iv) C₂-C₆ alkenyl substituted with oxo and C₁-C₃ alkoxy;
(v) C₁-C₆ alkyl substituted with ═NH;
(vi) C₂-C₃ alkenyl substituted with —OH;
(vii) C₈-C₁₂ alkenyl substituted with oxo and two C₁-C₃ alkoxy groups; and
(viii) aralkyl comprising a C₆-C₈ alkyl and a C₆ aryl, wherein the alkyl is substituted with oxo and the aryl is substituted with two alkoxy groups.

11. The compound of embodiments 8-10, in which R₂ is alkoxy.

12. The compound of embodiment 11, in which R₂ is C₁-C₃ alkoxy.

13. The compound of embodiment 12, in which R₂ is —OCH₃.

14. The compound of embodiments 8-13, in which R₃ is alkoxy.

15. The compound of embodiment 14, in which R₃ is C₁-C₃ alkoxy.

16. The compound of embodiment 15, in which R₃ is —OCH₃.

17. The compound of embodiment 8 or 9, in which:
R₁ is H;
R₂ is C₁-C₃ alkoxy;
R₃ is C₁-C₃ alkoxy; and
X is C₆-C₁₀ alkenyl, substituted with oxo, —OH, or two C₁-C₃ alkoxy groups;

18. The compound of embodiment 8 or 9, in which
R₁ is H;
R₂ is C₁-C₃ alkoxy;
R₃ is C₁-C₃ alkoxy; and
X is C₈-C₁₂ alkenyl substituted with oxo and C₁-C₃ alkoxy;

19. The compound of embodiment 8 or 9, in which
R₁ is OH;
R₂ is C₁-C₃ alkoxy;
R₃ is C₁-C₃ alkoxy; and
X is C₄-C₈ alkenyl substituted with two oxo groups.

20. The compound of embodiment 8 or 9, in which
R₁ is C₁-C₃ alkoxy;
R₂ is OH;
R₃ is C₁-C₃ alkoxy; and
X is C₄-C₈ alkenyl substituted with two oxo groups.

21. The compound of embodiment 8 or 9, in which
R₁ is H;
R₂ is OH;
R₃ is OH; and
X is C₁-C₆ alkyl substituted with ═NH.

22. The compound of embodiment 8 or 9, in which
R₁ is OH;
R₂ is C₁-C₃ alkoxy;
R₃ is C₁-C₃ alkoxy; and
X is C₂-C₃ alkenyl substituted with OH.

23. The compound of embodiment 8 or 9, in which
R₁ is H;
R₂ is C₁-C₃ alkoxy;
R₃ is C₁-C₃ alkoxy; and
X is C₈-C₁2 alkenyl substituted with oxo and two C₁-C₃ alkoxy groups

24. The compound of embodiment 8 or 9, in which
R₁ is OH;
R₂ is C₁-C₃ alkoxy;
R₃ is C₁-C₃ alkoxy; and
X is aralkyl comprising a C₆-C₈ alkyl and a C₆ aryl, wherein the alkyl is substituted with oxo and the aryl is substituted with two alkoxy groups.

25. The compound of embodiment 1, having a structure of Formula 2

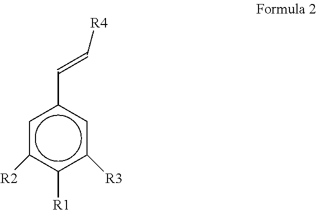

or a pharmaceutically acceptable salt thereof;

in which:
R₁ is H or OH;
R₂ is alkoxy or OH;
R₃ is alkoxy or OH; and
R₄ is C₁-C₁₅ alkyl, C₂-C₁₅ alkenyl, C₂-C₁₅ alkynyl, or aralkyl, each of which is substituted with at least one alkoxy, —OH, or oxo.

26. The compound of embodiments 25, in which R₄ is selected from the group consisting of:

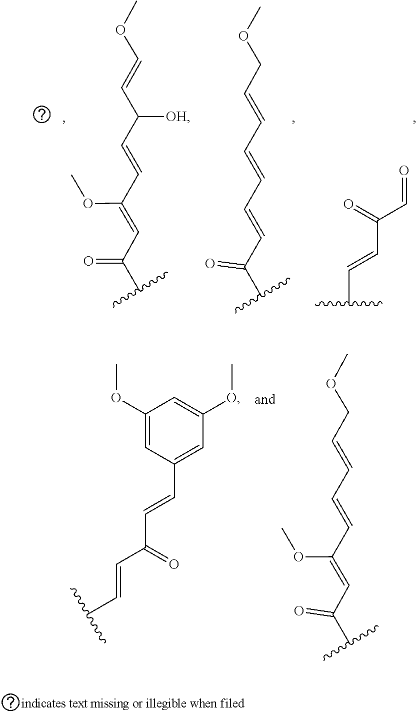 [SMILES: *C(=O)/C=C(/C=C/C(O)/C=C/OC)OC ; *C(=O)/C=C/C=C/C=C/COC ; */C=C/C(=O)C=O ; */C=C/C(=O)/C=C/c1cc(OC)cc(OC)c1 ; *C(=O)/C=C(/C=C/C=C/COC)OC]

27. The compound of embodiments 25 or 26, in which R₂ is alkoxy.

28. The compound of embodiment 27, in which R₂ is C₁-C₃ alkoxy.

29. The compound of embodiment 28, in which R₂ is —OCH₃.

30. The compound of embodiments 25-29, in which R₃ is alkoxy.

31. The compound of embodiment 30, in which R₃ is C₁-C₃ alkoxy.

32. The compound of embodiment 31, in which R₃ is —OCH₃.

33. The compound of embodiment 25 or 26, in which
R₁ is H;
R₂ is alkoxy;
R₃ is alkoxy; and
R₄ is

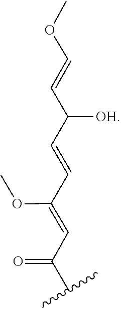 [SMILES: *C(=O)/C=C(/C=C/C(O)/C=C/OC)OC]

34. The compound of embodiment 25 or 26, in which
R₁ is H;
R₂ is alkoxy;
R₃ is alkoxy; and
R₄ is.

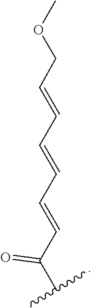 [SMILES: *C(=O)/C=C/C=C/C=C/COC]

35. The compound of embodiment 25 or 26, in which
R₁ is OH;
R₂ is alkoxy;
R₃ is alkoxy; and
R₄ is

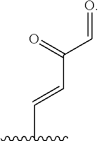 [SMILES: */C=C/C(=O)C=O]

36. The compound of embodiment 25 or 26, in which
R₁ is OH;
R₂ is alkoxy;
R₃ is alkoxy; and
R₄ is

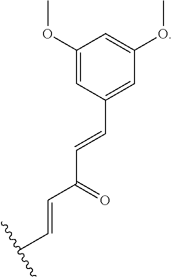 [SMILES: */C=C/C(=O)/C=C/c1cc(OC)cc(OC)c1]

37. The compound of embodiment 25 or 26, in which
R₁ is H;
R₂ is alkoxy;
R₃ is alkoxy; and
R₄ is

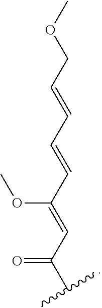

38. The compound of embodiment 1 having a structure of Formula 3

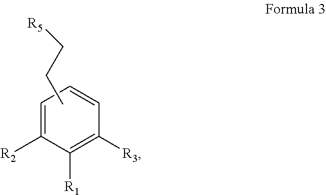

or a pharmaceutically acceptable salt thereof,

in which:
R₁ is H, alkoxy, or OH;
R₂ is alkoxy or OH;
R₃ is alkoxy or OH; and
R₅ is C₁-C₁₂ alkyl or C₂-C₁₂ alkenyl, which is independently substituted with 1, or 2, substituents selected from the group consisting of ═NH and oxo.

39. The compound of embodiment 38, in which R₅ is ═NH.

40. The compound of embodiment 38 or 39, in which R₅ is in the ortho position to R₂.

41. The compound of any one of embodiments 38-40, in which R₂ is OH.

42. The compound of any one of embodiments 38-41, in which R₃ is OH.

43. The compound of any one of embodiments 38-42, in which R₁ is H.

44. The compound of embodiment 38, in which R₅ is:

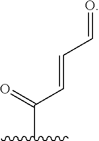 [SMILES: *C(=O)/C=C/C=O]

45. The compound of embodiment 38 or 44, in which R₃ is alkoxy.

46. The compound of any one of embodiments 38, 44, or 45, in which R₃ is C₁-C₃ alkoxy.

47. The compound of any one of embodiments 38 or 44-46, in which R₃ is —OCH₃.

48. The compound of any one of embodiments 38 or 44-47, in which R₁ is alkoxy.

49. The compound of any one of embodiment 38 or 44-48, in which R₁ is C₁-C₃ alkoxy.

50. The compound of any one of embodiment 38 or 44-49, in which R₁ is —OC₂H₅.

51. The compound of any one of embodiments 38 or 44-50, in which R₂ is —OH.

52. The compound of any one of embodiments 44-51, in which R₅ is in the para position to R₁.

53. The compound of embodiment 1, having a structure of Formula 4;

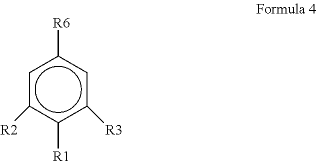

or a pharmaceutically acceptable salt thereof;

in which:
R₁ is OH;
R₂ is C₁-C₃ alkoxy;
R₃ is C₁-C₃ alkoxy; and
R₆ is C(CH₂)OH.

54. The compound of embodiment 53, in which
R₁ is OH;
R₂ is OCH₃;
R₃ is OCH₃; and
R₆ is C(CH₂)OH.

55. A pharmaceutical composition, comprising a compound of any one of embodiments 1-54 and a pharmaceutically acceptable carrier.

56. A method of treating a chronic disorder in a patient in need thereof, comprising administering a compound of embodiments 1-54 or a pharmaceutical composition of embodiment 55.

57. The method of claim 56, wherein the chronic disorder is acute lymphoblastic, acute lymphoblastic leukemia, acute lymphocytic leukemia, acute myelogenous leukemia, acute myeloid leukemia, adrenocortical carcinoma, AIDS-related lymphoma, anal cancer, appendix cancer, basal-cell carcinoma, bladder cancer, brain cancer, brainstem glioma, breast cancer, bronchial adenomas/carcinoids, Burkitt's lymphoma, carcinoid tumor, cerebellar or cerebral astrocytoma, cervical cancer, cholangiocarcinoma, chondrosarcoma, chronic lymphocytic or chronic lymphocytic leukemia, chronic myelogenous or chronic myeloid leukemia, chronic myeloproliferative disorders, colon cancer, cutaneous T-cell lymphoma, desmoplastic small round cell tumor, endometrial uterine cancer, ependymoma, esophageal cancer, Ewing's sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal tumor (GIST), gestational trophoblastic tumor, glioma of the brain stem, hairy cell leukemia, head and neck cancer, heart cancer, hepatocellular (liver) cancer, Hodgkin lymphoma, hypopharyngeal cancer, hypothalamic and visual pathway glioma, intraocular melanoma, Islet cell carcinoma, Kaposi sarcoma, laryngeal cancer, leukaemia, lip and oral cavity cancer, liposarcoma, lymphoma, male breast cancer, malignant mesothelioma, medulloblastoma, melanoma, Merkel cell skin carcinoma, mesothelioma, metastatic squamous neck cancer, mouth cancer, multiple endocrine neoplasia syndrome, multiple myeloma, multiple myeloma/plasma cell neoplasm, mycosis fungoides, myelodysplastic/myeloproliferative diseases, nasal cavity and paranasal sinus cancer, nasopharyngeal carcinoma, neuroblastoma, non-Hodgkin lymphoma, non-melanoma skin cancer, non-small cell lung cancer, oligodendroglioma, oral cancer, oropharyngeal cancer, osteosarcoma and malignant fibrous histiocytoma, ovarian cancer, ovarian germ cell tumor, ovarian epithelial cancer (surface epithelial-stromal tumor), ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia, pleuropulmonaryblastoma, primary carcinoma, primary central nervous system lymphoma, primary liver cancer, prostate cancer, rectal cancer, renal cell carcinoma, renal pelvis and ureter carcinoma, retinoblastoma, rhabdomyosarcoma, salivary gland cancer, Sezary syndrome, small cell lung cancer, small intestine cancer, soft tissue sarcoma, squamous cell carcinoma, stomach cancer, supratentorial primitive neuroectodermal tumor, testicular cancer, throat cancer, thymoma and thymic carcinoma, thyroid cancer, transitional cell cancer of the renal pelvis and ureter, urethral cancer, uterine sarcoma, vaginal cancer, visual pathway and hypothalamic glioma, vulvar cancer, Waldenstrom macroglobulinemia, Wilms tumor, Parkinson's disease and Parkinsonian disorders, Huntington's disease, Alzheimer's disease, multiple sclerosis, amyotrophic lateral sclerosis, Shy-Drager syndrome, progressive supranuclear palsy, Lewy body disease, spinal ischemia, spinal cord injuries, ischemic stroke, cerebral infarction, spinal cord injury, and cancer-related brain and spinal cord injury, multi-infarct dementia, geriatric dementia, other cognitive impairments, depression, Onychomycosis (fungal infection of the nail, Gingivitis and Periodontal Disease (Gum Disease) obesity, diabetes, SARS, COVID-19 or KRAS oncogene mutated cancer.

58. The method of embodiment 56 or 57, in which the chronic disorder is cancer.

59. The method of embodiment 58, in which the cancer is colon cancer, prostate cancer, breast cancer, or leukemia.

60. The method of embodiment 58 or 59, in which the cancer has a KRAS oncogene mutation. 61. The method of embodiment 56 or 57, in which chronic disorder is SARS or COVID-19.

### EXAMPLES

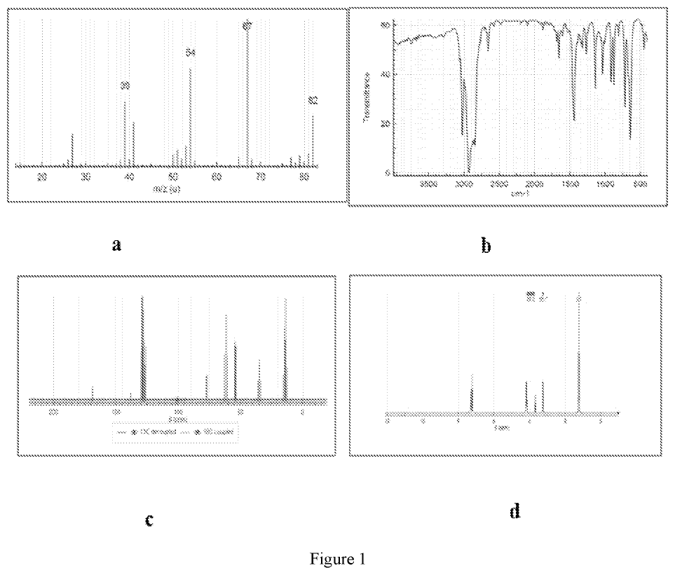

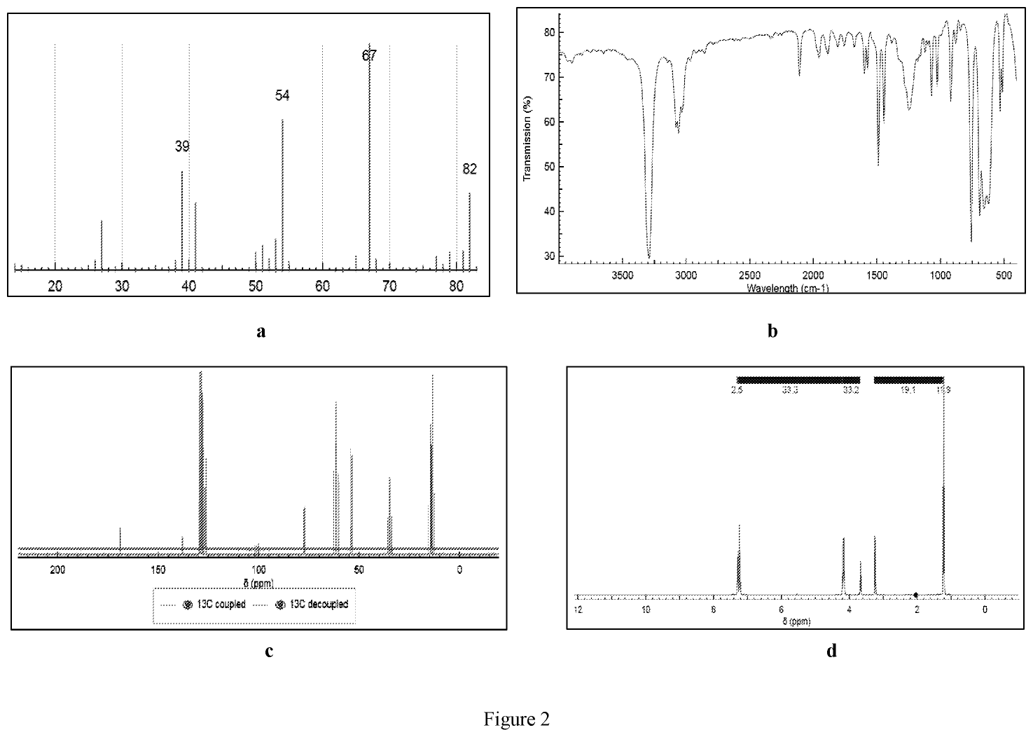

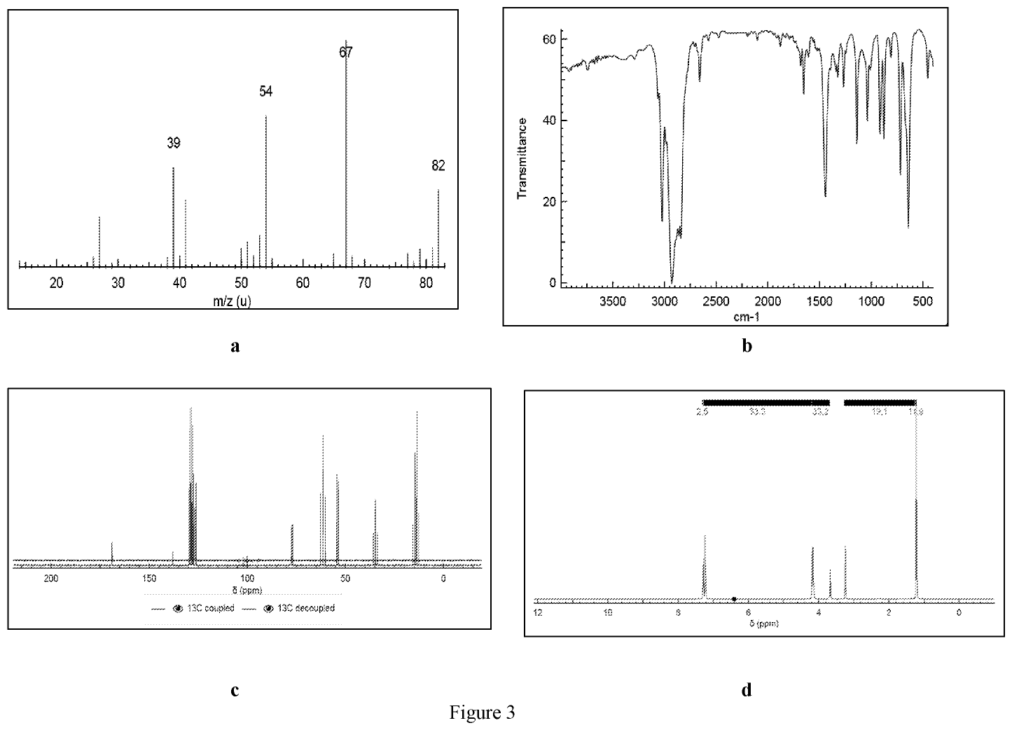

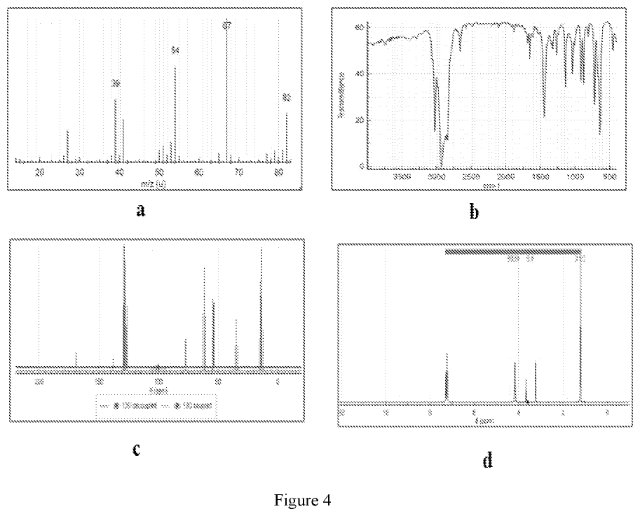

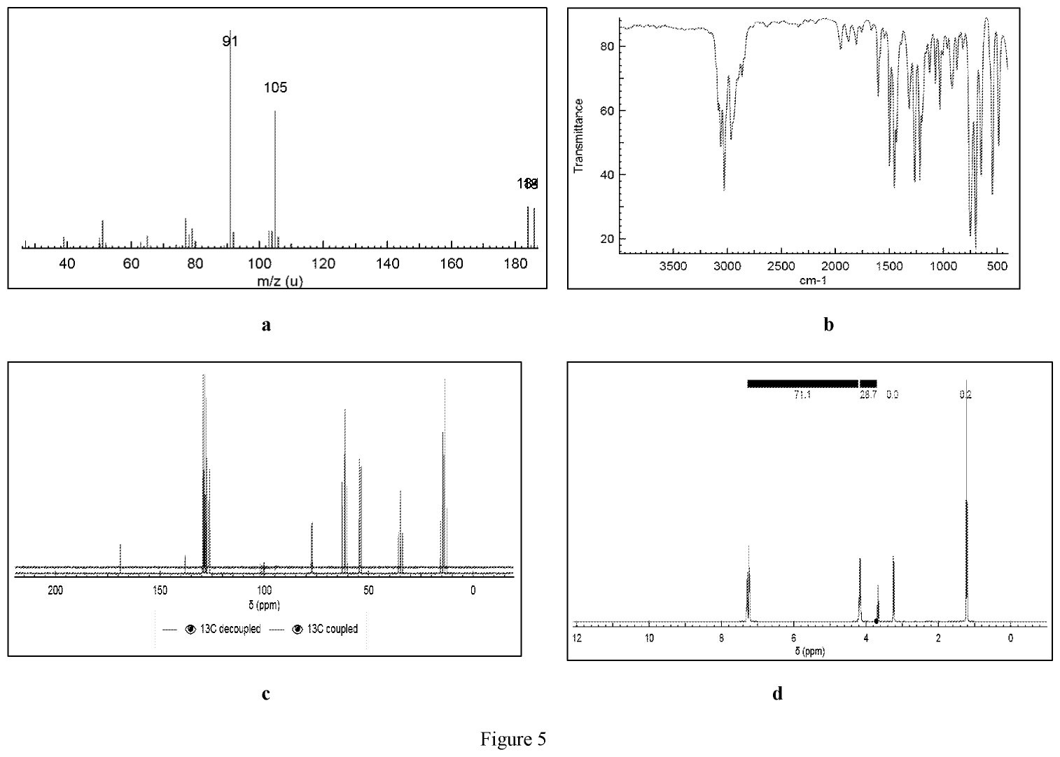

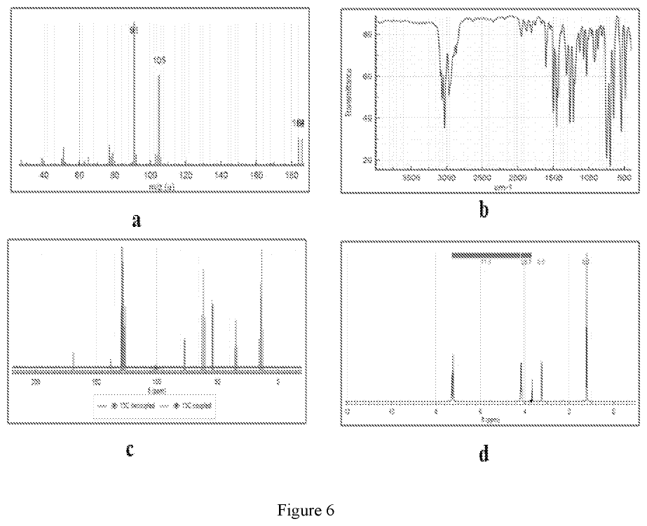

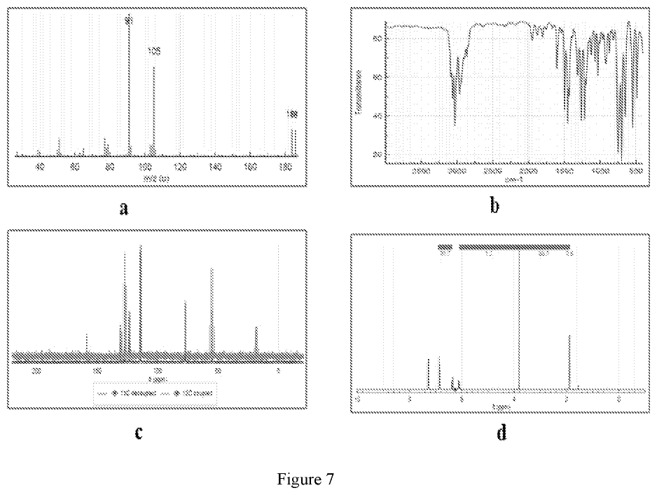

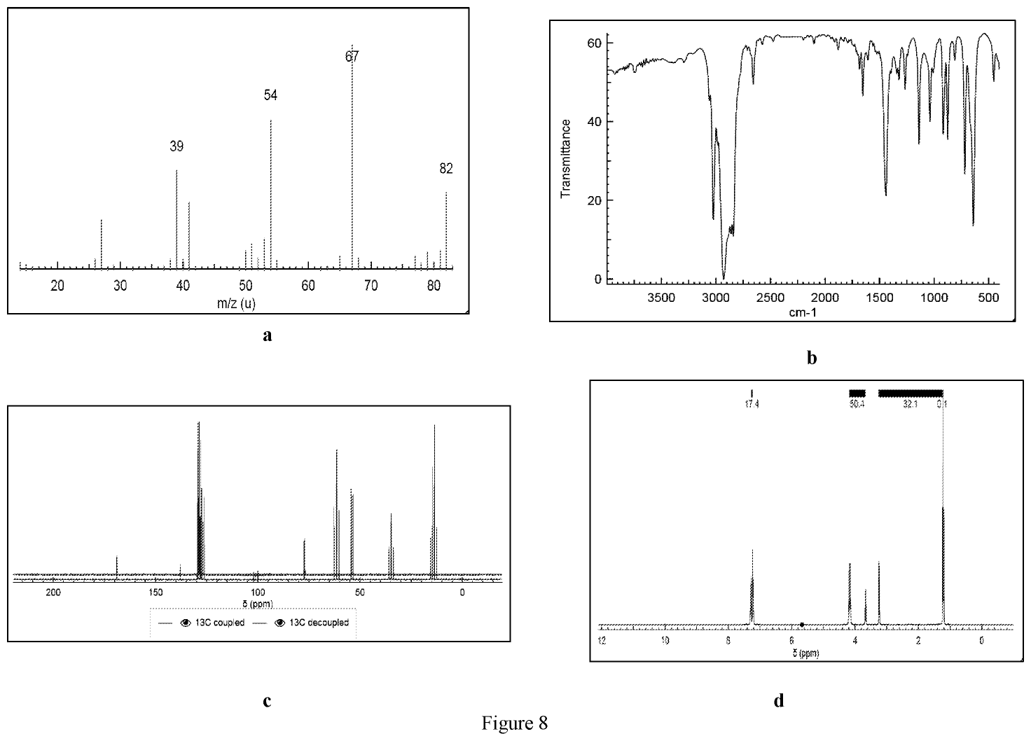

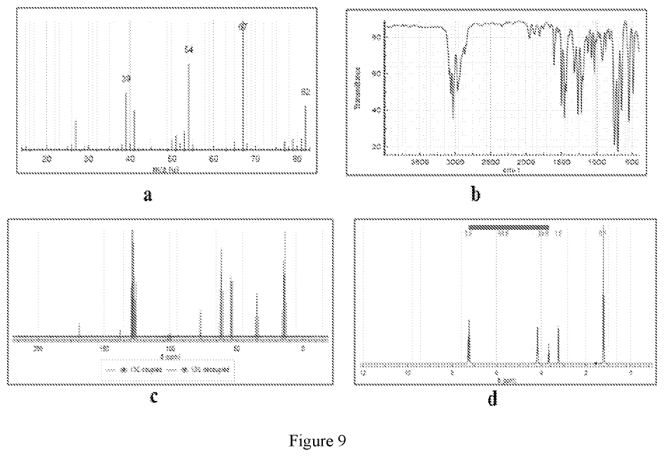

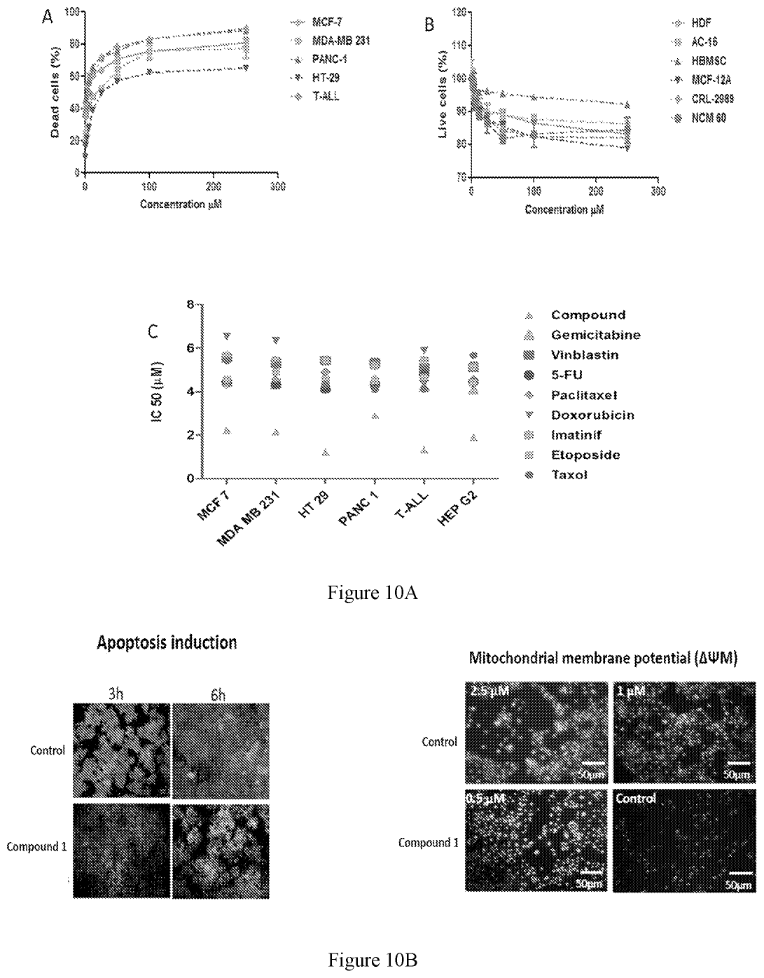

### Example 1. Anticancer Activity Studies (FIG. 10)

The synthesized compounds of Formula 1-4 were evaluated for cell proliferation, apoptosis, cell cycle arrest, the generation of reactive oxygen species and calcium were measured using MTT assay and flow cytometry, respectively. The expression of apoptosis- and proliferation-related proteins was determined by western blotting. The effect of molecules on apoptosis-related mRNA expression in cancer cells was detected by RT-PCR.

Molecules Induce Apoptosis and Cell Cycle Arrest in Pancreatic Cancer Cell Lines (PANC)

When Pancreatic adenocarcinoma cells (PANC) were treated with Compound I for 24 and 48 h at various doses (1-100 μg/mL), cell viability decreased significantly in time- and dose-dependent manners. Exposure of PANC cells to 10 μg/mL resulted in an approximate 65.5±0.88% decrease in viable cells compared with standard 5-Fluorouracil (10 μM) which showed 30.21±0.21% decrease in viable cells at 3 h. IC50 value was found to be 5.74±0.02 μg/ml and 5.21±0.19 μM of 5-FU after 24 hours of exposure. Most importantly, the molecules showed 85.29%±0.98 increases in protection towards normal pancreatic cells with an IC50 of 109.24 μg/ml whereas 5-FU showed 40.32±0.98% toxicity at 6 h. Changes in cell morphology were detected by the presence of cell membrane blebbing, chromatin condensation, and formation of apoptotic bodies (FIG. 10A).

In vitro studies show the disclosed compounds potentially target proteins involved in the G2/M checkpoint (FIGS. 10A and 10B), as it has been shown to induce arrest in this checkpoint in breast cancer, pancreatic cancer and other cancers which prevents cells from passing through this checkpoint and entering mitosis.

Cell growth inhibition was demonstrated by inducing apoptosis in a caspase-dependent manner through the intrinsic pathway (caspase-3 and 9) which resulted in subsequent loss of mitochondrial potential (ΔΨm) (FIG. 10B), increased ROS generation and DNA damage which consequently caused cell cycle arrest in G0/G1 phase in a dose- and time-dependent manner.

The novel molecules are believed to induce cell cycle arrest and senescent change, likely by targeting STAT-3, β-catenin/Wnt Signaling, MAPK, and/or JAK-1/2/3 aurora kinase A, thereby inducing mitotic arrest, altered expression of cell cycle-associated proteins, and disrupted microtubules.

### Example 2. Molecules Inhibit Histone Deacetylase (HDAC) Activity

Class I HDAC expression during pancreatic tumorigenesis was next examined. Briefly different concentration of Compound I were added and incubated for 24 h. Western blotting analysis confirmed that protein expression of Class I HDAC decreased in a dose-dependent manner compared to the control (p<0.05). Compound I significantly inhibited HDAC1, HDAC2, HDAC3, and HDAC8 from class I. Collectively; these data demonstrate that inhibition of class-I HDACs by molecules is sufficient to induce cell death in PANC cell lines. These results suggested that molecules is potent HDAC inhibitor. Inhibition of Class I HDACs is associated with the upregulation of histone H₃ acetylation and p21 mRNA and protein expression which have associated with the anti-proliferative activity. It has also been reported that HDAC6 functions as a α-tubulin deacetylase, modulating tubulin stability. Because α-tubulin and histone H₃ are mutual downstream targets of HDACs, the relationship between protein expression and function of molecules was further examined. The effects of Compound I on histone H₃ acetylation in PANC cells using western blot analysis was studied. Compound I induced stronger hyperacetylation of histone H₃ compared with control, which is constant with its potent inhibitory effect on class I HDAC1, but no acetyl-α-tubulin was detected.

Mechanism of Action (FIG. 11)

Without being bound by any particular theory, the compounds disclosed herein may exhibit one or more of the following mechanisms of action:
Induced cell cycle arrest and senescent change, probably by targeting, STAT-3 β-catenin/Wnt Signalling, MAPK, JAK-1/2/3 aurora kinase A, inducing mitotic arrest and altered the expression of cell cycle-associated proteins, and disrupted the microtubules.
Caused loss of mitochondrial membrane potential, cytochrome c release, up-regulation of Bax, downregulation of Bc1-2, and cleavage of caspase-3, thereby showing activation of mitochondrial-mediated apoptosis.
Multi-targeted kinase inhibitor that inhibits the proliferation of a variety of human cancer cells, cancer stem cells in vitro and in vivo through multiple pathways.
Histone deacetylase (HDAC) inhibitor and histone methyltransferase (HMT) inhibitor.
Downregulates the levels of HDAC2 and HDAC3 both at the mRNA and protein levels and also have the potential to downregulate KLF4 levels, which plays an important role in stem cell formation.
Downregulates hTERT levels, compared with standard drug suberoylanilide hydroxamic acid (SAHA) respectively.

To explicate the mechanism underlying the anti-proliferative effect, cell cycle distribution and apoptosis in PANC cells was investigated. Early apoptosis was observed by Annexin V-fluorescein isothiocyanate bound to the cell membrane as early as 6 hours. Compounds I-IX activated the intrinsic pathway of apoptosis by induction of caspase-3 and caspase-9. Involvement of the intrinsic pathway in the mitochondria could be seen, with a significant increase in mitochondrial permeability and cytochrome c release, whereas the mitochondrial membrane potential was decreased. Apoptosis was confirmed at the protein level, including Bax, Bc1-2, and survivin, while interruption of the cell cycle was used for final validation of apoptosis. Propidium iodide/Annexin V double staining revealed a pre-apoptotic cell population with PANC treated cells at 3 h. Furthermore, we observed that Fluorescence-activated cell sorting (FACS) analysis showed that the molecules induced cell cycle arrest is associated with modulation of the important checkpoint control proteins p21^{WAF1/KIP1}, p27^{KIP1}, p53 and cyclin A resulting in G0/G1-arrest in PANC cell lines, downregulated the expression of cyclin D3, cyclin E1, CDK2, CDK4, and CDK6 and upregulated the expression of p21, p27 and p53 via HDAC inhibition.

### Example 3. Molecules in Neurogenesis and Neuronal Development Human Umbilical Cord Stem Cell—Molecules Induced Neuronal Cell Type Formation (FIG. 12)

At the beginning of induction, HUMSCs cultures phenotypically exhibited large, thin flattened cell bodies, with large nuclei. MSCs that underwent treatment with molecules showed a change in morphology at 48-72 h. At this time, populations of MSCs exhibited spherical refractile cell bodies, with dendritic-like processes, and long thin axonal-like projections from the cell soma, typical of neurons. After treatment at 72 h, the ratio of neuron-like cells reached high, with the cell bodies contracted and the protuberances lengthened further. The MSCs retained this morphology for the duration of the induction.

### Example 4. KRAS Degradation

In brief, the present disclosure provides compounds which are capable of modulating G12C mutant KRAS, proteins. In some instances, the compounds act as electrophiles which are capable of forming a covalent bond with the cysteine residue at position of K-Ras4BG12C/G12D/G12V-GTP/GDP, K-Ras4BG13D-GTP/GDP, K-Ras4BQ61H-GTP/GDP, mutant protein. Methods for use of such compounds for treatment of various diseases or conditions, such as cancer, are also provided. Further mechanism study showed that compound AB-REV-001 can block the formation of the complex of guanosine triphosphate (GTP) and KRAS in vitro. In addition, AB-REV-001 inhibited KRAS downstream signaling pathway RAF/MEK/ERK and RAF/PI3K/AKT.

### Example 5. Cancer Stem Cell Inhibition

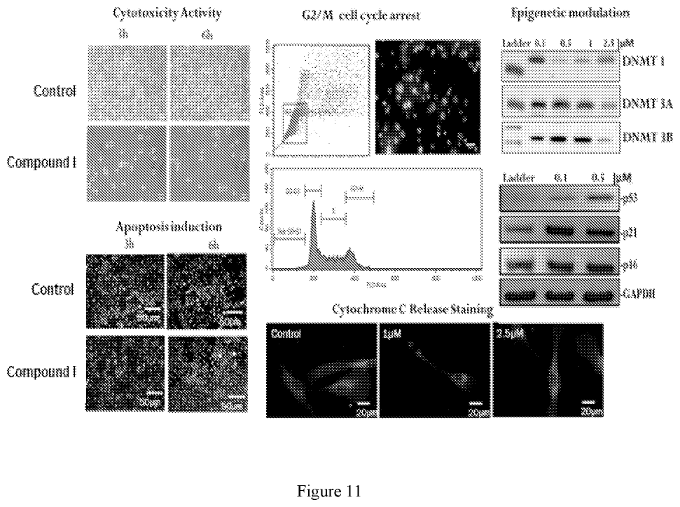

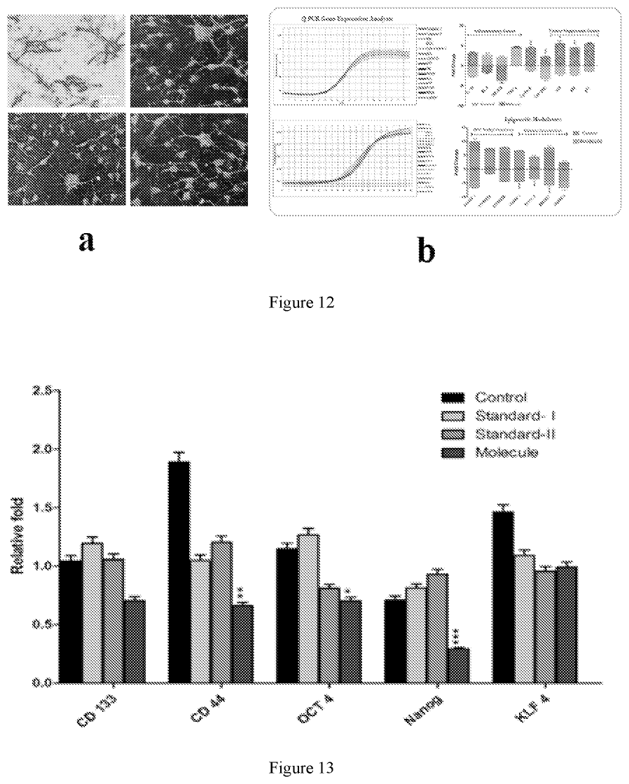

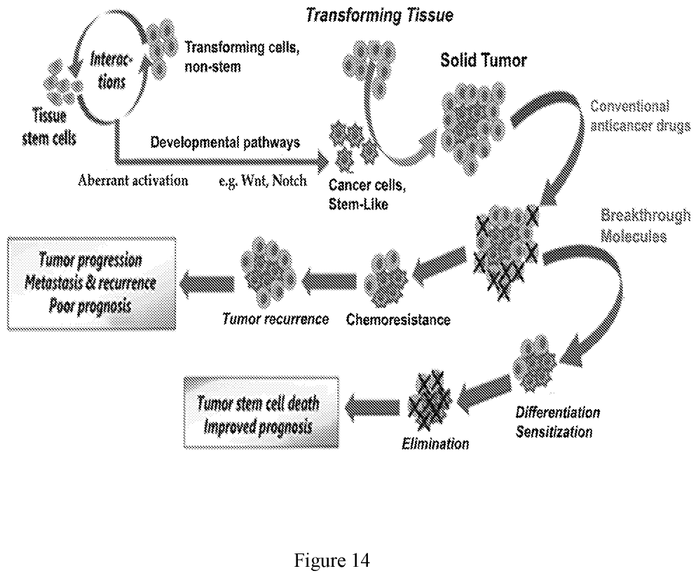

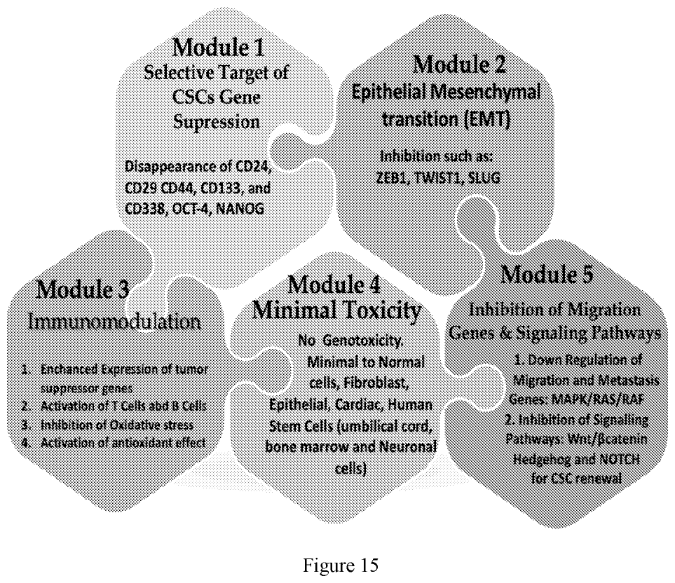

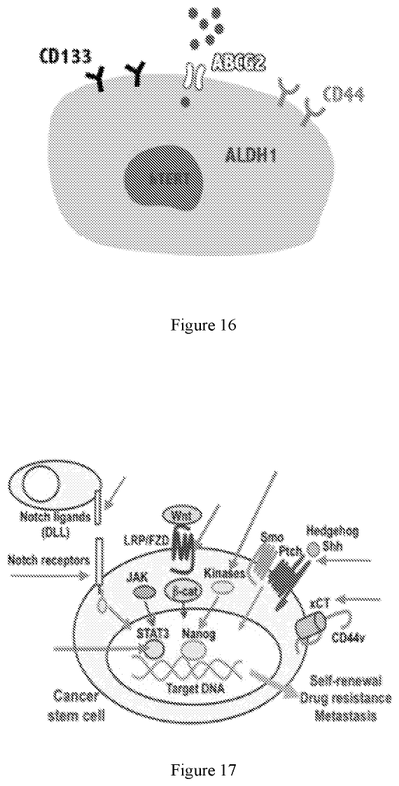

Cancer stem cells (CSCs) are believed to exhibit distinctive self-renewal, proliferation, and differentiation capabilities, and thus play a significant role in various aspects of cancer. CSCs have significant impact on the tumor progression, drug resistance, recurrence and metastasis in different types of malignancies. Conventional cancer chemotherapy often fails as most anti-cancer drugs are not effective against drug-resistant CSCs. These surviving CSCs lead to relapse and metastasis. Most studies report that conventional therapeutic agents have limited access to the CSCs due to their hypoxic microenvironment and distant location from the vasculature that retards the efficacy of anti-CSC drugs (FIG. 14). However, the disclosed compounds may overcome this limitation and may penetrate into a deeper location and destroy CSCs. Accordingly, in embodiments, the present invention provides a method of inhibiting cancer stem cell survival and/or self-renewal comprising administering to a cancer stem cell an effective amount of a compound disclosed herein, e.g., Compound I (FIG. 13).

FIG. 17 illustrates aberrant signal transduction pathways in CSCs and strategies for targeting CSCs. Signal transduction pathways in CSCs which play important roles in self-renewal, drug resistance, tumor recurrence and distant metastasis are being elucidated. The signaling pathways Notch, Wnt and Hedgehog signaling, and downstream effectors including the transcription factors β-catenin (β-cat), signal transducer and activator of transcription 3 (STAT3), and Nanog, play key roles of CSC properties. After interaction with xCT, a CD44 variant (CD44v) has enhanced capacity for glutathione synthesis and defense against reactive oxygen species (ROS). Due to this aberrant status, CSCs acquire the unique phenotype. An optimal method to eradicate CSCs is to identify the molecules responsible for the specific properties of CSCs, but not of normal cells. Target CSC phenotypes include: delta-like ligand (DLL), Frizzled (FZD), Janus kinase (JAK), lipoprotein receptor-related protein (LRP), Patched (Ptch), Sonic Hedgehog (Shh), and Smoothened (Smo).

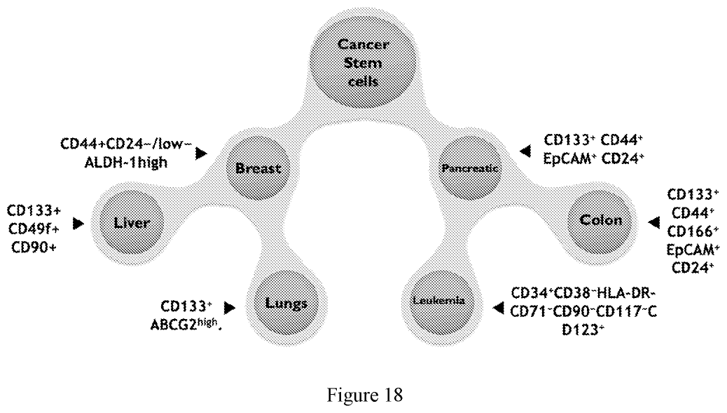

It has been demonstrated that the compounds of the present disclosure selectively target cancer stem cells in the tumor microenvironment and destroy CSCs by modulating the genes for self-renewal and differentiation. The compounds of the disclosure inhibited the expression of the genes in the follow cancers (FIG. 18):
Breast: CD44+CD24−/low Lineage−, ALDH-1high
Liver: CD133+, CD49f+, CD90+
Colon: CD133+, CD44+, CD166+, EpCAM+, CD24+
Pancreatic: CD133+, CD44+, EpCAM+, CD24+
Leukemia: CD34+CD38−
Lungs: CD133+, ABCG2^{High}
Leukemia: CD34+, CD38−, HLA−, DR−, CD71−, CD90−, CD117−, CD123+

Reduction of stem cell surface marker expressions are seen within 1 hour and up to a maximum of hours. No cancer stem cell markers are observed after 24 hours of treatment compared with other standard drugs.

### Example 6: In Vitro Cancer Studies with Compound I

Chemotherapy is a drug treatment that uses powerful chemicals to kill fast-growing cells in the body. However the treatment exhibits adverse side effects which could be simple gastritis and hair loss to serious bone marrow suppression, cardiac toxicity etc. The effect of compound I of the present invention in combination with chemotherapy drugs was investigated with respect to cytotoxicity. As detailed below, cytotoxicity of chemotherapy drugs such as Docetaxel, Paclitaxel, Pazobanib, Endoxon, Etoposide, Adriamycin, Dacromycin, Avastin, Gemcitabine, Cisplatin and Oxaliplatin were drastically reduced if cells were treated with Compound I of the present invention prior to treatment chemotherapy drugs.

The cytotoxicity of anticancer agents was studied using a panel of human normal cell lines alone and with the combination of Compound I of the present invention. The cytotoxicity effect was determined by MTT assay. The following cell lines were used: Human epidermal keratinocytes cells (HaCaT), Human dermal fibroblast (HDF), Human bone marrow mesenchymal stem cells (HBMSCs), Human normal hepatocytes cells (THLE2), Human cardiac cells (AC-16), Human intestinal epithelial cells (HIEC-6), Human neuronal cells (SHSY-5Y), Human vascular Endothelial cells (HuVECs), Human lung epithelial cells (Calu-3), Human lung fibroblast (MRC 5).

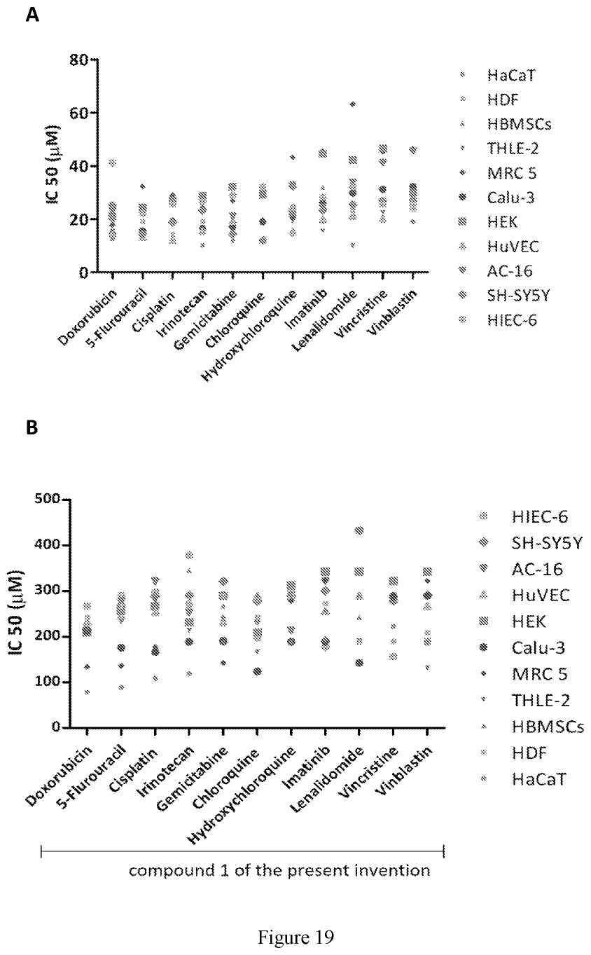

According to the data, there was a significant decrease in the normal cells and changes in morphology was observed, after treatment with different concentrations (1-500 μM) of anticancer agents compared with control p>0.05. The IC50 value was found to be average from 12.90 μM-40.50 μM for Doxorubicin, Gemicitanib, 5-FU, Cisplatin, Lenolidamide, Irinotecan, Chloroquine, Hydroxychloroquine, Vincristine and Vinblastin respectively (FIG. 19A). A significant level of apoptosis was observed in all the normal cell lines treated compared to negative controls which shows its distinct characteristic features.

Next, the cells were treated with anticancer agents in combination with Compound I of the present invention. After combination treatment, an increase in cell viability was observed, and no signs of cell rounding, granulation and cell shrinkage was observed, which shows that treatment of normal cells with Compound I reduces the toxic effect induced by anticancer agents. The IC50 was found to be increased compared with treatment of anticancer agent alone. Average of treatment across all cell lines shows IC50>100 μM, which shows the cytoprotective effect of Compound I (FIG. 19B). No significant apoptosis effect was observed in the combination study, which shows that Compound I reduces the cytotoxicity produced by the chemotherapeutic agents. No significant cytotoxic effect was observed in the normal cells lines, even at the highest concentration.

### Example 7: In Vivo Cancer Studies with Compound I

Metastasis Studies

In vivo studies indicated that tumor growth could be suppressed in xenograft nude mice models with an improved therapeutic window compared to standard drug treatment. Remarkably, no relapse or recurrence of tumor has been observed during the follow up studies 6 months later.

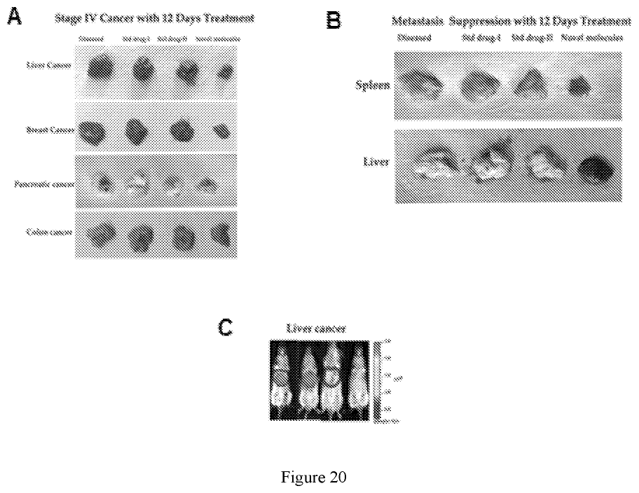

The compounds of the disclosure showed in vivo efficacy in triple-negative breast cancer, pancreatic cancer model, liver cancer model, and colon cancer model with a TGI (tumor growth inhibition) of 90% without any mortality growth inhibition in comparison to other standard drugs (FIG. 20).

In addition, the compounds of the disclosure significantly disrupted surrounding ECM organization, leading to increased quiescence, apoptosis, improved chemosensitivity, decreased invasion, metastatic spread and 6-fold reduction in tumor volume and cancer progression in vivo compared with Gemcitabine and 5-Fluorouracil treatment.

Further anti-cancer effects were demonstrated showing a significant inhibition of tumor recurrence in vivo when a single administration of the compounds of the disclosure was combined with standard drugs regimen.

Pancreatic Cancer Xenograft Model (PANC-1)

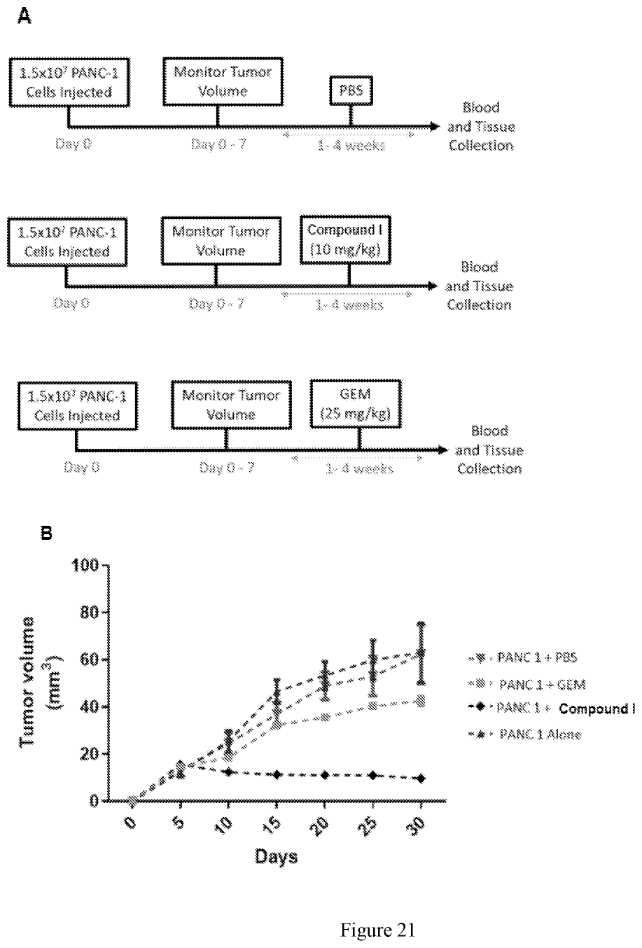

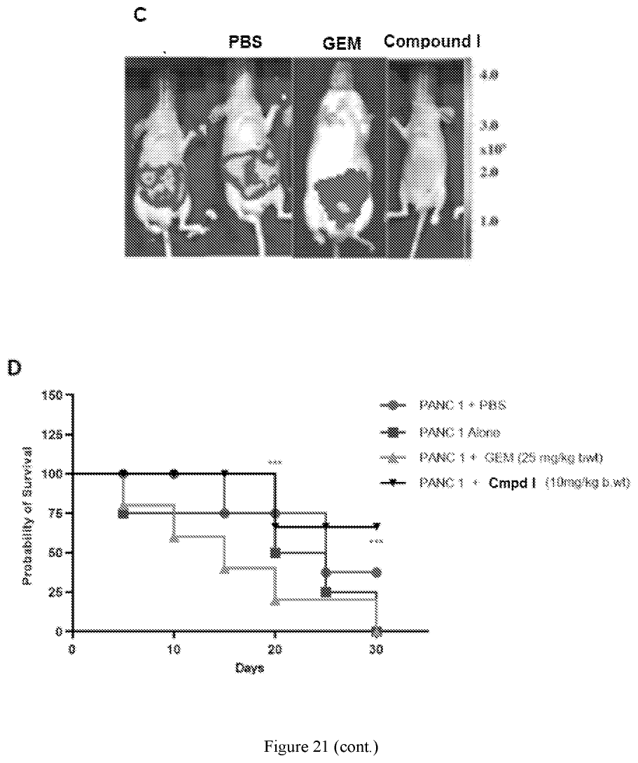

Study Design: A PANC-1 xenograft mouse model was carried out according to the experimental design described in FIG. 21A. Tumor volume was monitored over a 30-day period upon administration of PBS alone (vehicle control), 25 mg/kg gemcitabine, and 10 mg/kg Compound I.

Results: As shown in FIG. 21B, tumor volume steadily increases in mice (n=12) treated with 25 mg/kg gemcitabine from day 5 to day 30. In contrast, mice treated with 10 mg/kg Compound I saw tumor volume decrease over the same period. This result is confirmed by imaging studies that show no detectable tumor in mice treated with Compound I (FIG. 21C).

The reduction in tumor volume translated to improved survival in mice treated with Compound I (FIG. 21D).

Comparison to Standard Drugs

Compared to current treatment standard drugs (Table 1 below): Revlimid, Avastin, Herceptin, 5-Flurouraciland Gemcitabine, the compounds of the disclosure are 50-fold more potent in decreasing the number of tumorspheres and 100-fold more potent in reducing the CSCs population and prevented tumor relapse.

No significant toxicity and mortality were observed in the vital organs compared to standard drugs such as Revlimid (100% mortality), Avastin (60% mortality), Herceptin (60% mortality), 5-Flurouracil (70% mortality), and Gemcitabine (50% mortality) treating stage IV cancer.

Further the compounds of the disclosure when administered in combination with the standard drugs reduced the mortality rate and enhanced the sensitization of tumor cells with increased therapeutic efficacy.

TABLE 1
9 Breakthrough Small Molecules vs Standard Drugs

| S. | | | | Tumor | | Antioxidant | Mechanism of | | |
|---|---|---|---|---|---|---|---|---|---|
| No | Drugs | CSCs | Toxicity | volume | Metastasis | Activity | action | Regeneration | Mortality |
| 1 | 9 molecules | Specifically | No signs of | Significant | Decrease | Enhanced | Targets | Regeneration | No |
| | | target CSCs | toxicity, No | reduction | in | expression | WNT/β | of metastasis | mortality |
| | | and destroy | Cardiotoxicity, | in tumor | metastasis | of | Catenin, | organs due to | |
| | | CSCs | No | volume | genes, | antioxidant | Hedgehog | the | |
| | | population | Nephrotoxicity, | and | Activates | enzymes | Pathway | stimulation | |
| | | which inhibit | No | ingrowth | Tumor | and reduced | | of stem cells | |
| | | invasion and | hepatotoxicity | of tumor | suppressor | ROS levels | | has been | |
| | | extravasation | | | genes, | | | observed | |
| | | | | | Inhibition | | | | |
| | | | | | in EMT | | | | |
| 2 | Revlimed | No target | Cardiotoxicity, | No solid | No | No | TNF-related | No | 100 |
| | | specific | Hepatotoxicity, | tumor | prevention | significant | apoptosis | regeneration | |
| | | action of | Toxicity to vital | reduction | in | activity has | augmenting | of organs has | |
| | | stem cells | organs and | | metastasis, | been | the NK-T- and | been | |
| | | | normal tissues | | No | observed | NK-cell | observed | |
| | | | | | significant | | cytotoxicity | | |
| | | | | | changes in | | | | |
| | | | | | metastasis | | | | |
| | | | | | genes | | | | |
| 3 | Avastin | No specific | Toxic to vital | 20% | 10% | No | Inhibition of | No | 60% |
| | | target of | organs | Reduction | prevention | significant | VEGF | regeneration | |
| | | CSCs | | | | activity has | | of organs has | |
| | | population | | | | been | | been | |
| | | | | | | observed | | observed | |
| 4 | Herceptin | No specific | Hematological | 40% | No | No | Target HER2 | No | 60% |
| | | target of | and | changes | significant | significant | receptors | regeneration | |
| | | CSCs | gastrointestinal | has been | changes in | activity has | | of organs has | |
| | | population | toxicity | observed | metastasis | been | | been | |
| | | | | in tumor | genes | observed | | observed | |
| | | | | volume | | | | | |
| 5 | 5- | No specific | Cardiotoxicity, | 35% | No | 10% of | Thymidylate | No | 70% |
| | Flurouracil | target of | Hepatotoxicity | reduction | prevention | increased | synthase (TS) | regeneration | |
| | | CSCs | | in tumor | in | antioxidant | inhibitor | of organs has | |
| | | population | | volume | metastasis, | parameters | | been | |
| | | | | | No | has been | | observed | |
| | | | | | significant | observed | | | |
| | | | | | changes in | | | | |
| | | | | | metastasis | | | | |
| | | | | | genes | | | | |
| 6 | Gemcitabine | 5% Target on | Developed | There was | Prevention | No | Deoxycytidine | No | 50% |
| | | CSCs | signs of | no | in | significant | ana logue, a | regeneration | |
| | | population | systemic | significant | metastasis | activity has | pyrimidine | of organs has | |
| | | | toxicity | difference | genes and | been | antimetabolite | been | |
| | | | | in the | inhibition | observed | | observed | |
| | | | | tumor | in EMT | | | | |
| | | | | weight | | | | | |
| | | | | change | | | | | |

Down Regulation of Migration Genes and Signaling Pathways

In comparison to standard drugs which show limited access to CSCs due to a hypoxia environment, the compounds disclosed herein may prevent metastasis to the lungs and lymph nodes by inhibiting lymphangiogenesis and angiogenesis (VRGFR) in the pre-metastatic organs.

Without being bound by any particular theory, the multi-tyrosine-kinase inhibitor may induce apoptosis and suppress the invasiveness of cancer cells by inhibiting activated NF-κB, Akt, ERK2, Tyk2, and PKC. Without being bound by theory, the compounds also attenuated the migration and invasion through the inhibition of the PI3K/Akt/mTOR signaling pathway. Without being bound by any particular theory, the compounds effectively suppress metastasis, angiogenesis and invasion of cancer cells via ERK1/2−, Akt/NF-κB/mTOR- and p38 MAPK-dependent NF-κB signaling pathways.

Modulation of Epithelial Mesenchymal Transition (EMT)

All 9 compounds disclosed herein inhibited the tumor growth in xenograft mouse model and modulated the expression of mesenchymal and epithelial markers. They suppressed the expression of mesenchymal genes such as fibronectin, vimentin, N-cadherin, TWIST, and SNAIL, and increased expression of epithelial genes such as Occluding and E-cadherin via specifically targeting canonical WNT/β-catenin/Hedgehog, TGFβ/BMP-SMADs pathways.

Immunomodulation The compounds of the disclosure increased the number and activity of cytotoxic T cells and promoted the activation of macrophages, NK cells and DC which facilitate APC to CD4+ and CD8+ through capture, internalization, processing and presentation of tumor antigens via MEW class I and II molecules.

In vivo tumors treated with the molecules further exhibited reduced ECM deposition and impaired infiltration of CD31⁺ endothelial cells, α-SMA⁺ cancer-associated fibroblasts as well as F4/80⁺ macrophages suggesting that treatment created a suppressive tumor microenvironment, which in turn suppressed tumor growth, invasion and metastasis.

### Example 8: Targeting Lung Cancer Stem Cells

Compound I has a significant effect on targeting specific lung cancer stem cells and induces apoptosis and inhibits metastasis without harming the normal cells which aid in the treatment of cancer therapy. Compound I is orally bioavailable, multi-tyrosine-kinase inhibitor with 120 target proteins that induces apoptosis and suppresses the invasiveness of lung cancer stem cells with no toxicity to normal cells. There were no signs of discomfort or any cardiovascular or respiratory disorders observed in animals after the administration throughout the study period.

### Example 9: Compounds of the Present Disclosure as Inhibitors ACE-2 and Nsp15

Objective: To determine if compounds of the present disclosure, e.g., Compound I, are potential inhibitors of ACE-2 and Nsp15, which are proteins responsible for entry and replication of viruses such as SARS-CoV-2 in human cells.

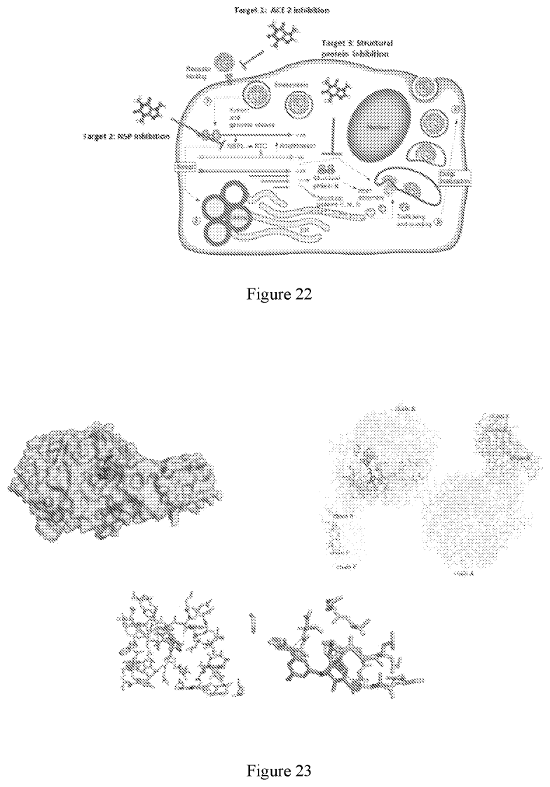

Background: To infect a human host, viruses must be able to gain entry into individual human cells. Viruses use host cell machinery to produce copies of themselves, which then spill out and spread to new cells. Studies have demonstrated that SARS-CoV-2 attaches to the target cells by an interaction between its spike protein (S) and host cell protein angiotensin converting enzyme-2 (ACE-2), a transmembrane enzyme found on the surface of a cell. This interaction on the host cell surface is of significant interest since it initiates the infection process (FIG. 22). Accordingly, drugs modulating the biological activity of ACE-2 were suggested as potential candidates for treatment of this viral infection.

Another protein identified from Severe Acute Respiratory Syndrome Coronavirus 2 (SARS-CoV-2) that may play a role in virus progression is Nsp15 (FIG. 22). Nsp15 is 89 percent identical to the protein from the earlier outbreak of SARS-CoV. An analysis of SARS-CoV revealed that inhibition of Nsp15 can slow viral replication. The newly mapped protein is conserved among coronaviruses and is essential in their lifecycle and virulence. Initially, Nsp15 was thought to directly participate in viral replication, but more recently, it was proposed to help the virus replicate possibly by interfering with the host's immune response.

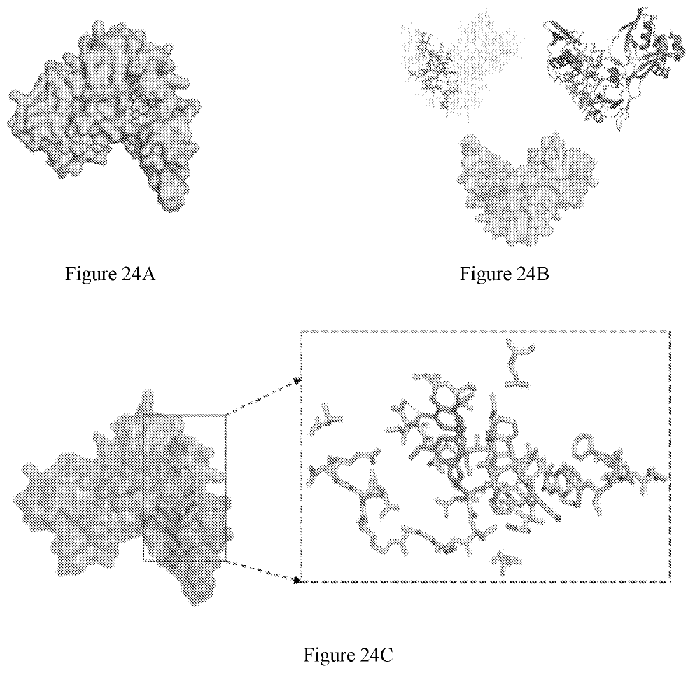

Results: To evaluate the potential of compounds of the present disclosure to target ACE-2 and Nsp15, studies were conducted using an in silico approach and molecular docking. From these analyses, Compound I was found to have a high binding affinity with all the viral proteins tested (FIGS. 22-24). Specifically, FIG. 21 shows the affinity of Compound I for the ACE-2 binding site, which results in a disruption of the interaction between the virus and the receptor. Compound I was also found to have affinity for the Nsp15 binding site, as shown by FIGS. 24A-C.

Summary: The present disclosure provide small molecule inhibitors that target the S-ACE-2 mediated entry of SARS-CoV-2 entry into human cells. In addition, the compounds of the present disclosure also target Nsp15, which can slow viral replication. Therefore, compounds of Formula 1-4 have potential for development as effective drugs against COVID-19.

### Example 10: In Vitro Antiviral Activity of Compound I in Vero Cells

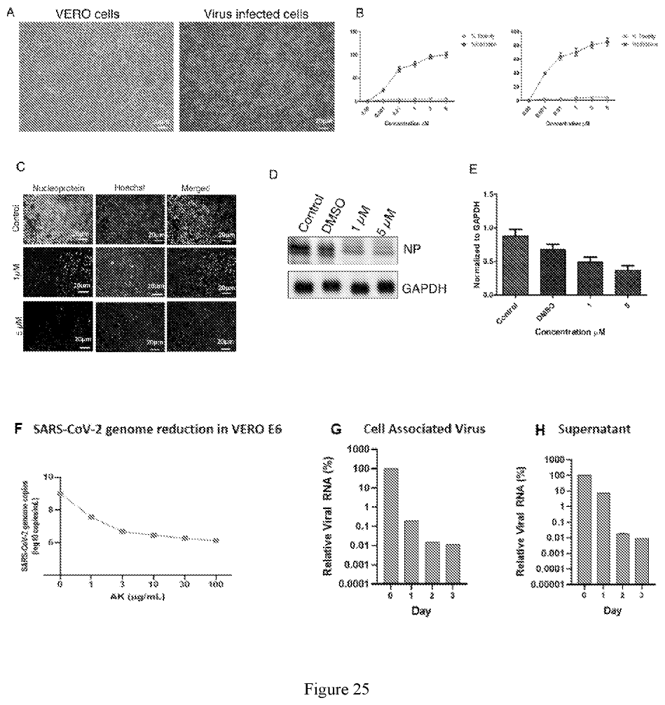

Study Design: Standard assays were carried out to measure the effects of Compound I on the cytotoxicity, virus yield and infection rates of SARS-CoV-2 (FIG. 25). First the cytotoxicity of Compound I was tested on Vero cells. Then the Vero cells were infected with SARS-CoV-2 (FIG. 25A) at a multiplicity of infection (MOI) of 0.1 PFU/cell in the presence of varying concentrations of Compound I. DMSO was used as vehicle. Efficacies were evaluated by quantification of viral copy numbers in the cell supernatant via quantitative real-time RT-PCR (qRT-PCR) and confirmed with visualization of virus nucleoprotein (NP) expression through immunofluorescence analysis at 48 h post infection (p.i.) (FIG. 25C). In addition, post infection, cells were exposed to the test compound to further estimate its potential as a prophylaxis and treatment against coronaviruses. Also, the protective effect of Compound I in lung epithelial (Calu-3) cell lines was evaluated.

Antiviral activity and cytotoxicity: To test the antiviral activity of Compound I, Vero cells were infected with SARS-CoV-2 isolate at the MOI of 0.1 for 2 h (FIG. 25B), followed by the addition of different concentrations of Compound I (0.001, 0.01, 1, 3, 5 μM, respectively). From an evaluation of the data obtained from dose-response curves, it was found that Compound I potently exhibits antiviral activity with an IC50 of 1 μM (FIG. 25B, blue line, which represents mean inhibition of virus) and CC50 of >15 μM (FIG. 25B, red line, which represents cytotoxicity of drug).

To further determine the effectiveness of Compound I, cells infected with SARS-CoV-2 were treated with serial dilutions of Compound I and 2 h post infection both supernatant and cell pellets were collected for real-time RT-PCR (FIGS. 25F-25H).

At 24 h, there was a 100% reduction in viral RNA present in the supernatant (indicative of released virions) of samples treated with Compound I compared to the vehicle DMSO. Similarly, a 99% reduction in cell-associated viral RNA (indicative of unreleased and unpackaged virions) was observed with Compound I treatment. At 36 h this effect increased to the reduction of viral RNA in Compound I treatment, which shows statistical significance when compared to control samples, indicating that Compound I treatment resulted in the effective loss of essentially all viral material by 36 h.

A reduction in viral RNA was observed in both supernatant and cell pellets from samples treated with 1 μM Compound I at 48 h, equating to a 99% reduction in viral RNA in these samples compared to the control samples. Again, no toxicity was observed with Compound I at any of the concentrations tested.

Immunofluorescence microscopy of virus-infected cells upon treatment of Compound I was carried out. Specifically, Vero cells were infected with SARS-CoV-2 at MOI of 0.1 and treated with 1 μM and 1 μM of Compound I. At 48 h p.i., the infected cells were fixed, and then probed with rabbit sera against the NP of SARS-related CoV as the primary antibody and Alexa 488-labeled goat anti-rabbit IgG as the secondary antibody, respectively. The nuclei were stained with Hoechst dye. Bars, 20 μm. Staining results show that viral load measured by nucleoprotein expression was substantially reduced in Vero cells treated with either dose of Compound I (FIG. 25C). These results were confirmed by Western blot analysis of infected cells 24 h p.i. (FIG. 25D) and quantified by normalization to GAPDH (FIG. 25E).

### Example 11: In Vitro Activity of Compound I in Lung Epithelial Cells

The antiviral activity of Compound I was assessed against SARS-CoV-2 in Calu-3 cultures. A dose-dependent reduction in replication approaching was observed at 0.1-5 μM and as compared to untreated controls with average IC50 values of 0.01 μM (SARS-CoV-2). To determine if Compound I could inhibit replication, we first evaluated antiviral activity and cytotoxicity in the continuous human lung epithelial cell line Calu-3 cells. Compound I inhibited SARS-CoV-2 replication in cells with an average half-maximum effective concentration (IC50) value of 1.5 μM (FIG. 25B). Importantly, we did not observe any cytotoxicity at concentrations up to 100 μM thus demonstrating the 50% cytotoxic concentration (i.e. CC50) for Compound I is in excess of 100 μM in Calu-3 cells. Taken together, these results suggest substantial inhibition of SARS-CoV-2 replication will be achieved at low micromolar concentrations in lung epithelial cells.

### Example 12: The Impact of Compound Ion the Stages of Viral Infection

It was hypothesized that Compound I would inhibit SARS-CoV-2 at early stage replication by inhibiting viral RNA synthesis. To test this hypothesis and determine which stage in the viral replication cycle Compound I inhibited SARS-CoV-2, Vero cells were infected with a multiplicity of infection (MOI) of 0.1 PFU/cell, resulting in a single-cycle infection, and treated them with 1 μM Compound I at 2 h intervals from 2 h pre-infection to 10 h post infection. Maximal inhibition was observed when Compound I was added between 2 h pre-infection and 2-h post infection. Less inhibition was detected when Compound I was added between 6 and 8 h post infection, and no inhibition was observed when Compound I was added after 10 h post infection. These results demonstrate that Compound I inhibits SARS-CoV-2 at early stages of infection. Because viral RNA is synthesized early in infection and Compound I is implicated in inhibiting viral RNA synthesis, the cellular level of viral RNA was next determined by real-time quantitative PCR (qPCR) after treatment with Compound I. Treatment with increasing concentrations of Compound I resulted in decreased viral RNA levels that correlated with the decrease in titer was observed. These results suggest that Compound I inhibits SARS-CoV-2 early after infection by interfering with viral RNA replication.

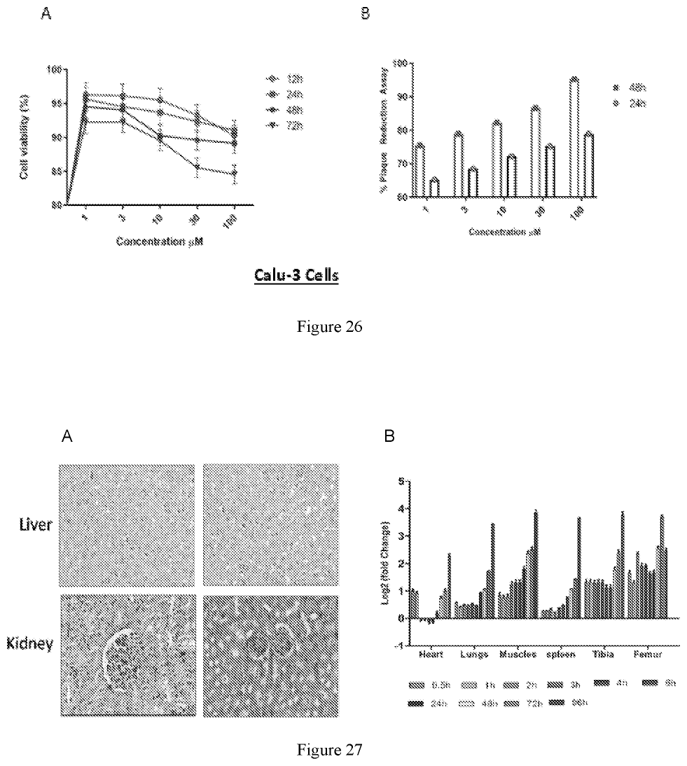

In the model of normal epithelial cells SARS-CoV-2 infection in human lung epithelial cells (calu-3 cell line), Compound I was investigated to see if it also is able to inhibit SARS-CoV-2 replication in human cells. The results indicated that Compound I is able to inhibit SARS-CoV-2 replication at the same concentrations as in VERO cells (IC 50 10 μM Compound I treated cells =<102 TCID50/ml), demonstrating that the antiviral activity of Compound I is not dependent on the type of cells (FIG. 26A). As for other coronaviruses, the expression of the SARS-CoV genome is mediated by translation of the genomic RNA and a ‘nested’ set of subgenomic messenger RNAs, produced by a unique mechanism involving discontinuous transcription during RNA synthesis. To determine whether, as in the case of SARS COV-2, Compound I was acting by blocking viral RNA synthesis, Calu-3 cells were infected with SARS-CoV-2 and treated with different concentrations (1.5 and 10 μM) of Compound I soon after the virus adsorption period. Total RNA was extracted 24 h p.i. and analyzed by RT-PCR. As shown in FIG. 26B, Compound I treatment caused a decrease of intracellular SARS-CoV-2 RNA levels dose-dependently, reaching an inhibition of more than 95% of control at concentrations of Compound I that did not affect RNA synthesis in uninfected cells. Viral genomic RNA was quantitated in clarified supernatants by quantitative reverse transcription polymerase chain reaction (qRT-PCR;). Like the effect on infectious titers, a dose-dependent reduction was found in viral genomic RNA and a similar calculated IC50 of 5 Collectively, these data demonstrate that Compound I is potently antiviral against two genetically distinct emerging CoV.

### Example 13: Immune Response of Compounds of the Present Disclosure

Compound I has the ability to activate CD4+ helper 7, cells and CD8+ cytotoxic I-cells and generates an immune response in the body to protect against viral infections, Specifically, Compound I increases the number and activity of cytotoxic cells and promotes the activation of macrophages NK cell and DC which facilitate APC to CD4+ and CD8+ through capture, internalization, processing and presentation of antigens via MHC class I and II molecules.

### Example 14: Proposed Mechanism of Action in the Lungs

In a xenograft stage 4 metastasized breast cancer animal model, Compound I decreased production of cytokines (tumor necrosis factor alpha [TNF-α] and interleukin-6 [IL6]) and chemokines (CXCL10, CCL2, CCL3, CCL5), and were correlated with migration of Natural Killer cells and macrophages and observed in the lungs. From data collected by QPCR gene expression studies, by day 7, histopathological evidence showed normal lung pathology without any pneumonitis observed and a decreased expression of cytokines (INF-α, IL-6, gamma interferon [IFN-γ], IL-2, and IL-5), chemokines (CXCL9, CXCL10; CCL2, CCL3, and CCL5), and receptors (CXCR3, CCR2, and CCR5) was detected in the lungs, associated with an influx of T lymphocytes, There were no observed signs of clinical illness and histopathological evidence of disease characterized, by bronchiolitis, interstitial pneumonitis, diffuse alveolar damage and fibrotic scarring.

### Example 15: Potentiating Hydroxychloroquine by Reducing Side Effects in Treatment of COVID-19

Coronavirus disease (COVID-19) is an infectious disease caused by virus SARS-CoV-2. The disease causes respiratory illness with symptoms such as a cough, fever, and in more severe cases, difficulty breathing. At present, one drug that may be effective in treating COVID-19 is hydroxychloroquine. However the drug hydroxychloroquine causes serious side effects including cardiovascular diseases, eye damage, mild or severe bronchospasm, and also affects mental health. The effect of Compound I of the present invention in reducing the side effects associated with hydroxychloroquine was studied according to methods known in the art. It was found that Compound I of the present invention assists in potentiating hydroxychloroquine by reducing the side effects in treatment of COVID-19 (FIGS. 20 and 21).

### Example 16: Pharmacokinetics (PK), Tissue Distribution, and Toxicity

The pharmacokinetics and toxicity of Compound I was studied in vivo in a nude mouse model.

PK Study:

An analysis of the data shows the mean plasma concentration of Compound I was significantly higher in comparison with the control group throughout the entire 6 h sampling period. Following intravenous administration, Compound I was widely distributed in several tissues, including the hippocampus, heart, lung, stomach, liver, mammary gland, kidney, spleen, femur, and tibia (FIG. 27A-27B). There was a 29-fold increase in the maximum plasma concentration (Cmax) and a 28-fold increase in the area under the curve (AUC) for the treatment group as compared to the control group (p>0.05).

Significantly Compound I was widely distributed in several organs, and especially those with high porosity, consistent with its pharmacodynamic activities in these organs. In addition, the volume of distribution of Compound I after IV dosing was found to be significant when compared to control, which implies a good tissue distribution. The tissue distribution in the current study showed that Compound I at 10 mg/kg IV reaches appropriate levels for pharmacodynamic activities in the hippocampus, femur, tibia, and mammary gland, which are related to the prevention and therapeutic target for neurodegenerative diseases and other chronic diseases (FIG. 27B). No toxicity was observed.

Tissue-to-plasma ratios were taken of Compound I in animals at 1, 2, 4, 24 h after administration. At 60 min, the highest levels of Compound I were found in the lungs, brain, stomach, liver, mammary gland, and small intestine, all of which are highly perfused organs followed by spleen and heart. Up to 72 h, Compound I was detected in most organs, but not in the femur and kidney. The ratios in most organs continued to increase up to 4 h. Interestingly, the tissue-to plasma ratio of Compound I in the hippocampus and brain increased significantly and continuously from 1, 2, and 24 h after dosing. No signs of discomfort or any cardiovascular or respiratory disorders were observed in animals after the administration and throughout the study period. There was no change in body weight monitored for a 7-day period after drug administrations.

PK Results: Pharmacokinetics studies with the compounds disclosed herein revealed prolonged persistence in systemic blood circulation and no nephrotoxicity, cardiotoxicity and hepatotoxicity was observed compared with other standard drugs, which show severe cardiotoxicity hepatotoxicity, gastrointestinal toxicity and respiratory disorders.

Summary: The novel molecules of the present disclosure have shown enhanced pharmacokinetics, biodistribution and tolerability when compared with standard drug administration. In vivo biodistribution studies revealed that accumulation of novel molecules in the tumor of the animal model was considerably higher (P<0.01) than in the other organs analyzed.

Acute Oral Toxicity Study:

Experimental Design: The assay of acute toxicity was performed according to the Organization for Economic Cooperation and Development (OECD) guideline 423 (OECD, 2001a). A total of 60 mice weighing between 27 and 37 g were randomly divided into six experimental groups of 10 mice each (5 males and 5 females per group). After fasting overnight Compound I was administered to each treatment group at single doses of 200, 500 1000, 2000, mg/kg, respectively, by oral gavage. The control groups were treated with the same volume of distilled water. After dosing, all animals were observed individually for mortality and changes in general behavior during the first 30 min, then at 2, 4, 6, 10 and 24 h following treatment. Symptoms of toxicity such as hypo-activity, piloerection, breathing difficulty, tremors, and convulsion were evaluated after administration of the various doses. The LD 50 value was determined according to the method described by the OECD Guidelines 423 (OECD, 2001a). During the remaining experimental period, the animal observation was performed at least once per day for the post-dosing period of 14 days. Body weights were measured at the initiation of treatment, and on days 4, 7, 11 and 14 after administration. On the 14th day, the mice were sacrificed under anesthesia, and vital organs (heart, kidneys, lung, spleen and liver) were removed for macroscopic examination.

Sub-toxicity Study:

Experimental Design: The sub-chronic toxicity study was carried out according to OECD Test Guidelines 408 for testing chemicals (OECD, 2008). A total of 48 male and female Wister rats weighing between 170 and 240 g were randomly divided into four groups (n=6 males and 6 females/group). Rats in treatment groups orally received Compound I at doses of 200, 500, 1000 and 2000 mg/kg/day. Compound I was administered by oral gavage at 10 mL/kg body weight on a daily basis for 28 days. Rats in control groups were administrated orally with the same volume of distilled water (vehicle). During the experimental period, the body weights of all groups were measured once a week. Animals were also visually observed for mortality, changes in behavioral patterns, changes in physical appearance, and symptoms of illness. At the end of the treatment period, all rats fasted overnight (12-16 h), and then anesthetized with urethane by intraperitoneal injection (1 mL/100 g body weight). Blood samples were collected for the measurement of hematological (EDTA-2K coated tubes) and biochemical (dry tubes) parameters. After euthanasia, the rats were sacrificed and organs were removed for necropsy, organ weight measurement, and histopathological examination.

Urinalysis: On the last week of the treatment period, a urine test was conducted in all rat groups. Fresh urine was collected overnight from all animals, to determine specific gravity, pH, levels of leukocytes, nitrites, protein, glucose, ketones, blood, urobilinogen, and bilirubin. Urine samples were analyzed using an automatic urine analyzer and test strips.

Hematology and serum biochemistry: For the hematological investigation, all animals fasted overnight but were allowed access to water ad libitum. The rats were then anesthetized, and blood samples were collected from the abdominal aorta. Whole blood was collected in EDTA tubes (containing potassium salt of ethylenediamine-tetracetic acid) and processed immediately for hematological analysis. The parameters measured were red blood cell count (RBC), hematocrit (HCT), hemoglobin (HGB), mean corpuscular volume (MCV), mean corpuscular hemoglobin concentration (MCHC), mean corpuscular hemoglobin (MCH), white blood cell count (WBC), neutrophils (NEU), eosinophils (EOS), basophils (BASO), lymphocytes (LYM), and monocytes (MONO). The hematological analysis was performed using an automatic hematological analyzer. For the measurement of biochemical parameters, dry tubes containing collected blood were centrifuged at 3000 rpm at 5° C. for 15 min to obtain the serum. Serum samples were analyzed using an automated biochemistry analyzer. The clinical biochemistry parameters included, total serum protein (TP), albumin (ALB), total bilirubin (T-BIL), alkaline phosphatase (ALP), alanine aminotransferase (ALT), aspartate aminotransferase (AST), uric acid (URIC), urea (UREA), creatinine (CREA), low-density lipoprotein-cholesterol (LDL-C), high-density lipoprotein-cholesterol (HDL-C), total cholesterol (TC), triglycerides (TG), and glucose (GLU). Serum electrolytes such as calcium (Ca2+), sodium (Nat), potassium (K⁺), and chloride (CF) were also determined.

Necropsy and organ weight: All groups of rats were subjected to gross necropsy, which included the examination of the thoracic organs, external surface, and all of the internal organs. Vital organs were carefully examined macroscopically for any type of abnormalities. Thereafter, various organs, including the heart, liver, kidneys, stomach, lung, spleen, adrenals, thymus, epididymis, testes, uterus, and ovaries were surgically removed, cleaned with ice-cold saline solution, placed on absorbent papers, and then weighed (absolute organ weight in grams). The relative organ weight (ROW) of each animal was then calculated as follows: ROW=[Absolute organ weight (g)÷Bodyweight of rat on sacrifice day (g)]×100.

Histopathology: The major organs (lung, heart, liver, kidney) and reproductive organs (testis and ovary) were removed for histopathological examinations. After weight measurement, the organs were quickly fixed in 10% buffered formalin (pH 7.4). Following fixation, tissue specimens were dehydrated in a graded series of ethanol (70-100%), cleared in toluene, and finally enclosed in paraffin. Thereafter, 5-μm thin sections were prepared using a microtome (Leica) and stained with Haematoxylin and Eosin (H & E) prior to microscopic examination. The microscopic features of the organs of treated groups were compared with the control group, and photomicrographs were recorded.

Statistical analysis: All data are expressed as the means±standard deviation (SD). Statistical significances between control and treated groups were determined by one-way analysis of variance (ANOVA), followed by Dunnett's post hoc test. Graph Pad Prism version 6.0 for Windows was used for statistical analysis. Data analyses from male and female groups were done separately, and the differences were considered statistically significant at p<0.05.

Toxicity Results:

General Signs and Behavior Analysis
The clinical signs and symptoms are the crucial observations to monitor the toxicity effects of drugs on organs (Jothy et al., 2011). In our present study, no treatment related death was observed in animals of both sexes for acute and subacute toxicity studies at the specified oral dose of Compound I.
During the 14 (acute) and 28-day (subacute) observation period, animals did not demonstrate any adverse changes in physical behavior, food, and water consumption.
No gross or macroscopic abnormalities were observed in all animals of both (acute and subacute toxicity) the groups. Therefore, median lethal dose (LD50) of the drugs can be considered to be greater than 2,000 mg/kg.
Substances having LD50>2,000 mg are considered as relatively safe according to Globally Harmonised Classification System (GHS) (Miyagawa, 2010). Hence, Compound I can be classified as Category 5 according to GHS.

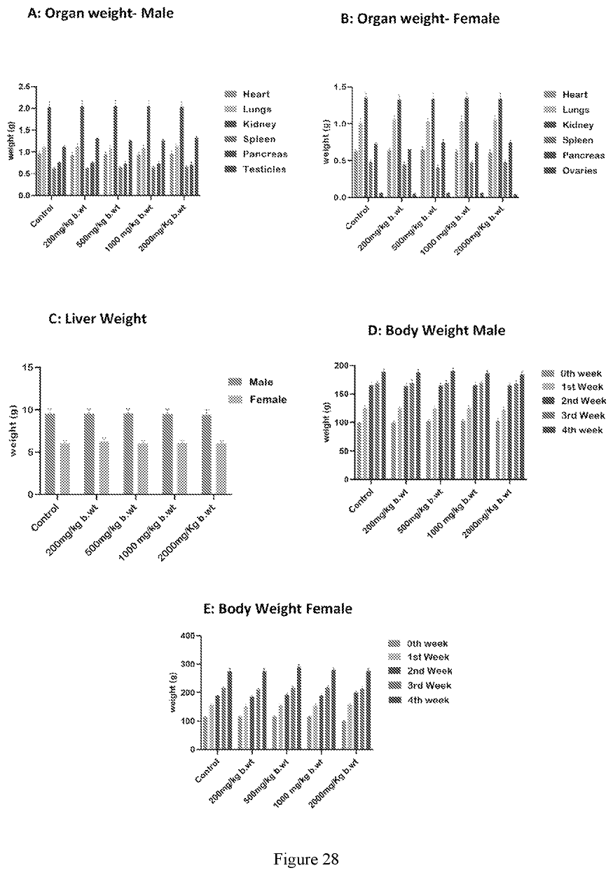

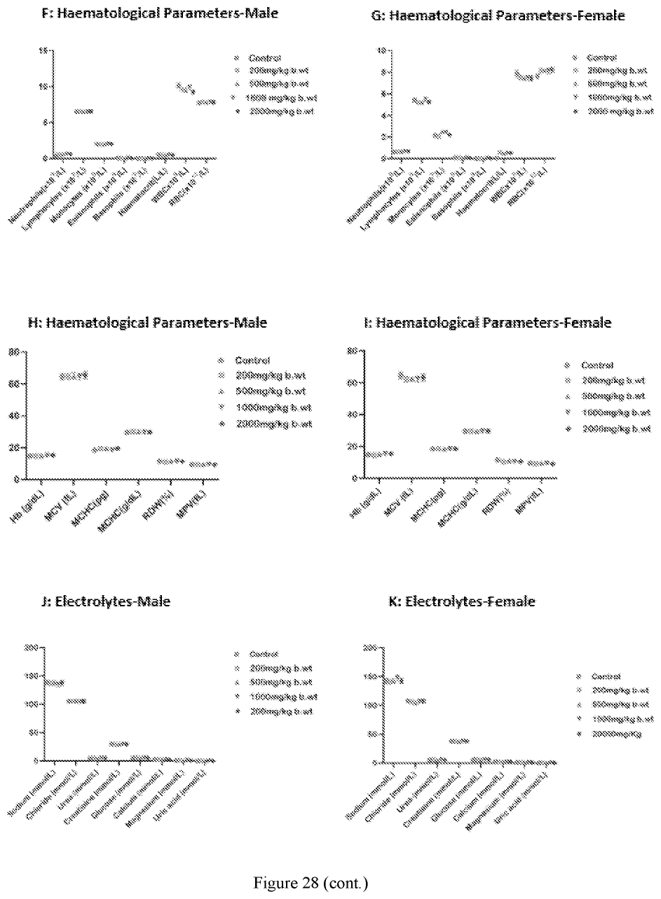

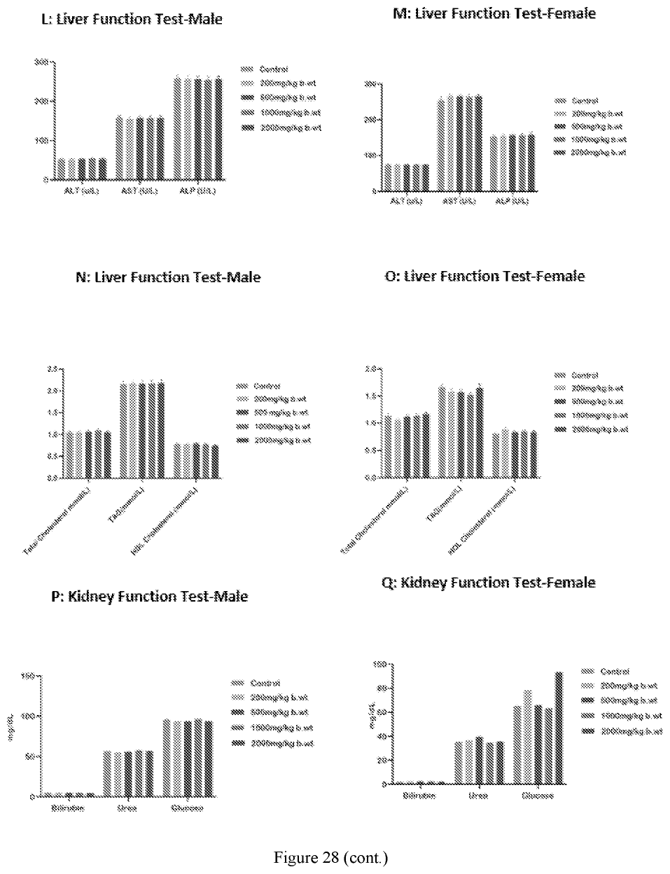

Effect of Compound I on Body Weight and Organ Weight (FIGS. 28A-28E)
Exposure to potentially toxic drug will cause a drastic fall in body weight gain of the rats (Teo et al., 2002). Alterations in body weight and relative organ weight are indicators of toxicity and wellness assessment in experimental animals (Piao et al., 2013).
The body weight of the control and treated rats were shown in FIG. 28D-28E. In this study, all rats at each dosage group showed sustained weight gain during the experimental periods which indicates that Compound I did not elicit any deleterious effect on body weight in both the acute and subacute toxicity groups.
Moreover, no significant difference in the percentage increase of body weight was compared between the treated and control groups.

Relative Organ Weight (ROW)
Relative organ weight (ROW) of the liver, brain, kidney, heart, spleen, of both the tests were shown. Differences between the ROW of control and treated groups were statistically important, when evaluated against control group.
There were no significant changes has been observed in all the organs and thus it can be established that the administration of Compound I did not elicit any adverse effects to the vital organs.

Effects of Compound I on Food and Water Intakes
FIG. depicts the effects of the Compound I on the food and water intakes in subacute treatment.
The single daily administration of Compound I at study doses for 28 d caused no significant changes (P>0.05) in food and water intakes when compared with the control group.

Effect of Compound I on Hematological Parameters (FIGS. 28F-28I).
Hematological parameters have a crucial role in establishing the toxicity induced by the drugs (Petterino and Argentino-Storino, 2006). Alterations in blood parameters have a superior predictive assessment of human safety evaluation, when the data is translated from experiments on laboratory animals (Olson et al., 2000). The evaluation of hematological parameters is of great importance in determining the health status of an individual.
The reference value for RBC, WBC, PCV, MCH, and MCHC are 7-10×10\hat{ }6/μl, 6-18×10\hat{ }3/μl, 35%-64%, 14.3-19.5 pg, and 26.2-40 g/dl, respectively. In the current investigation, average value of the hematological parameters of both the groups (acute and subacute toxicity study), such as WBC, RBC, PCV, and hemoglobin of the treated groups, were not significantly altered when compared to the control group (Loha et al., 2019).
This result suggests Compound I may not have any toxic substances that can lead to conditions like anemia or other abnormalities.
The increased release of WBCs is a notable biomarker of stress and also aids in defending the body against some inflammatory conditions, such as bacterial infections, leukemia and haemorrhage.
The result obtained from this study revealed that Compound I did not cause any significant changes in the level of WBC count, or in their subtypes, including neutrophils, lymphocytes, monocytes and eosinophils, at any of the doses, relative to control. This suggests that the Compound I is nontoxic.

Effect of Compound I on biochemical parameters (FIGS. 28L-28Q)
The biochemical evaluations are of vital importance to evaluate safety of drugs on the hepatic and renal function. The data on biochemical parameters of control and treated groups is presented on FIGS. 28L-28Q. In this study, all the measured biochemical parameters of the acute and subacute study group did not show any significant changes.
In the current work, potential hepatotoxicity of Compound I was assessed by measuring the enzymatic activities of aminotransferases (ALAT and ASAT)— see FIGS. 28L-28M.
An abnormal increase in aminotransferase activities (ALAT and ASAT) could frequently refer to hepatotoxicity [Fortson et al., 1985].
The results showed that the biochemical parameters of treated group with a dose up to a maximum (2000 mg/kg body) were not directly affected when compared to the control (p>0.05).
These findings were in agreement with those of acute toxicity, which showed no clinical symptoms nor behavioural changes occurred in mice treated with similar doses.
However, there is no effect on the activity of aminotransferase of treated group when compared to the control group (p>0.05).
The insignificant changes in plasma levels of AST, ALT, ALP activities at the doses of 200, 500, 1000 and 2000 mg/kg in both sexes of the animals are clear indications that the Compound I caused no damage to the liver.

Renal Function Test (FIGS. 28J-28K)
The kidney function was also assessed for potential toxic effects induced by the drug by measuring urea and creatinine concentration since any significant change in these parameters could refer to induced-nephrotoxicity [Mukinda et al., 2010; Gnanamani, A et al., 2008].
The retention of creatinine, electrolytes, urea and uric acid in the body is indicators of kidney damage. Alteration in the levels of some electrolytes such as Na+, K+, Cl− and Mg2+ can also be a sign of renal injury.
Our findings revealed that there was no significant difference in the level of creatinine, electrolytes, urea or uric acid in the all the dose, when compared with the control group, in both sexes of the rats and also Compound I had no effect on serum electrolytes (Na+, Ca2+, Mg2+ and C₁—). (FIGS. 28J-28K).
Furthermore, there was no significant difference in the levels of total protein, albumin, conjugated bilirubin and bilirubin when compared with the control group.
This provides further support for the safety of Compound I at these doses, as there was no alteration in kidney function.

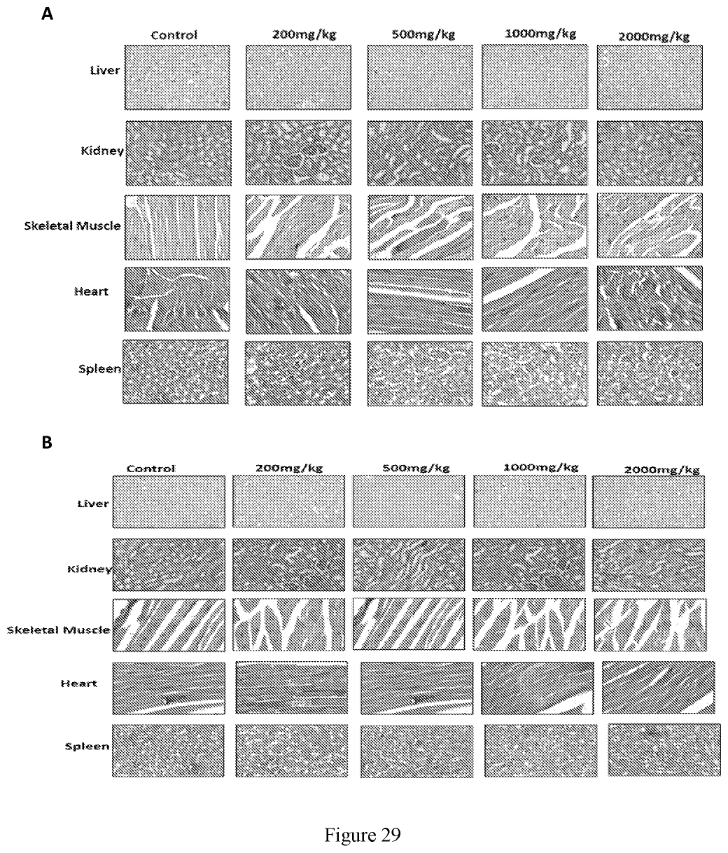

Histopathological studies (FIGS. 29A-29B)
Histopathological analysis of the organ samples of liver, kidney, pancreas, heart, lungs, stomach, and reproductive organs from both male and female rats were performed on the final day of the treatment period and the results for several of these tissues are tabulated in FIGS. 29A-29B.

Liver:
Multiple sections of the male liver showed normal hepatocytes along with normal portal triads, sinusoidal spaces and central venous system in Compound I treated group.
Sections of female rat liver from the treated groups showed almost normal cellular architecture with normal hepataocytes. There was also normal appearance of the portal triad including hepatic portal vein, interlobular bile duct, and branches of hepatic artery. Liver sections of both male and female rats from the control group showed normal liver architecture.

Kidney:
Multiple sections taken out from renal biopsy of both male and female rats of the treated groups showed almost normal size and shape of glomeruli, tubules, intestinum and blood vesicles.
There was no strong evidence of acute tubular necrosis and glomerular changes for the Compound I treated groups. The renal biopsy sections of the control group rat both male and female showed normal findings.

Pancreas (not shown):
Sections of both male and female rat's pancreas showed normal architecture in the control treated group, whereas in Compound I treated group almost no abnormalities were observed in the architecture of both pancreatic acini and islets.

Heart:
Sections of the heart taken from both male and female rats appeared normal in control treated as well as Compound I treated rats, however in male rats no significant changes has been observed.

Lungs (not shown):
In both male and female rats, multiple sections of lungs showed normal cellular architecture, alveoli, and lymphatic vessels in the control treated group. In the Compound I treated groups no lymphocytic infiltrations were observed for both male and female rats.

Stomach (not shown)
Sections of the stomach from both sexes of rats showed normal findings in the control treated groups. In the Compound I treated group, female rats showed normal cellular architecture with normal mucosa, submucosa, muscularis externa and serosa, whereas in male rats showed normal cellular architecture with no polyp formation or hyperplastic changes were observed.

Reproductive Organs (not shown)

Sections of the reproductive organs i.e. testis for male and ovary for female showed normal pathology for both the control and Compound I treated groups.

### Example 17: Anticipated Recovery of COVID-19 Patient after Administration of Compound I

Dosage: PO administration of 250 mg of Compound I, B.I.D. over 7 days

Day 01:

The orally bioavailable drug Compound I is absorbed in the intestine and distributed throughout the body. It targets the SARS-CoV-2 viral entry, inhibits replication and proteolytic processing, and shuts down the protein production machinery of the virus into the host cells.

Day 02:

Compound I stimulates the body's own immune system by acting as an immunomodulator, increasing the production of anti-inflammatory cytokines and interferon, and inhibiting lysosomal activity in host cells which target against the viral infections. An elevated level of circulating IL-6 will be decreased which is associated with a controlled level of lung elasticity and protection from more severe bronchoalveolar inflammation.

Day 03:

The induced immune response inhibits further virus replication, promotes virus clearance from the respiratory tract, induces tissue repair and triggers prolonged adaptive immune response against the viruses which reduced the disease progression. After treatment, the peripheral lymphocytes will be increased, the C-reactive protein decreased, and the overactivated cytokine-secreting immune cells (CXCR3+CD4+ T cells, CXCR3+CD8+ T cells, CXCR3+NK cells) will be decreased within 3-5 days, which decreases the cytokine storm induced by SARS-CoV-2.

Days 04-06:

Collectively, treatment with Compound I inhibits the SARS-CoV-2 virus particles which invade the respiratory mucosa and infect other cells, which in turn triggers a series of immune responses and the production of a cytokine storm in the body that may be associated with the critical condition of COVID-19 patients. The pulmonary function and symptoms of the patients will be improved within 4-5 days after Compound I administration and could lessen lung injury caused by excessive immune response to SARS-CoV-2.

### SUMMARY

Compound I has shown significant inhibitory effects on many key proteins from similar coronaviruses such as SARS-CoV (FIG. 22). This compound inhibits viral enzymes including proteases ACE-2 receptor and viral replication protein Nsp15, thereby greatly reducing infection and replication of the virus within the host cell. In vivo studies in mice further show no toxicity to normal cells and healthy, normal functioning of animals after 9 months since administration with no relapse of disease. Without being bound by theory, it is believed that the molecules of the present invention have the ability to activate CD4+ helper T cells and CD8+ cytotoxic T cells and generate an immune response in the body to protect against viral infections.

Furthermore, a dose dependent inactivation of SARS-CoV-2 was observed upon direct exposure to the Compound I and 50% reduction (IC50) was achieved at 1 μM. Together, the data supports further development of Compound I for treatment of CoVs and suggests a novel mechanism of Compound I interaction with the CoV replication complex that may shed light on critical aspects of replication. As explained above, a pharmacokinetic study investigated the concentrations of Compound I at different time intervals after either oral administration or intravenous injection. In tissue distribution, Compound I was found to be mainly concentrated in the heart, lungs, liver and other organs. In the gastrointestinal tract, a significant amount of Compound I accumulated in the stomach, implying that Compound I may be absorbed through stomach tissue and distributed throughout the body. In addition, less than 2% Compound I was excreted in either urine or feces, indicating that approximately 98% of Compound I was either absorbed or distributed to the vital organs. Collectively, our current findings will provide a more complete understanding of the biological actions of Compound I in vivo.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1-2. (canceled)

3. A compound as claimed in claim 1, having a structure of Formula 1 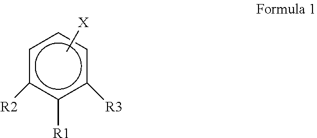
or any derivative thereof, pharmaceutically acceptable salt thereof, or combination thereof
wherein:
R₁ is H, alkoxy, or OH;
R₂ is alkoxy or OH;
R₃ is alkoxy or OH; and
X is C₁-C₁₅ alkyl, C₂-C₁₅ alkenyl, or aralkyl, chain, each of which is independently substituted with at least one alkoxy, OH, ═NH, or oxo group.

4. The compound as claimed in claim 3 wherein X is:
(i) C₆-C₁₀ alkenyl, substituted with four substituents independently selected from the group consisting of oxo, —OH, and C₁-C₃ alkoxy;
(ii) C₈-C₁₂ alkenyl substituted with oxo and C₁-C₃ alkoxy;
(iii) C₄-C₈ alkenyl substituted with two oxo groups;
(iv) C₂-C₆ alkenyl substituted with oxo and C₁-C₃ alkoxy;
(v) C₁-C₆ alkyl substituted with ═NH;
(vi) C₂-C₃ alkenyl substituted with —OH;
(vii) C₈-C₁₂ alkenyl substituted with oxo and 2 C₁-C₃ alkoxy groups; and
(viii) aralkyl comprising a C₆-C₈ alkyl and a C₆ aryl, wherein the alkyl is substituted with oxo and the aryl is substituted with two alkoxy groups.

5. The compound as claimed in claim 3, wherein:
R₁ is H;
R₂ is C₁-C₃ alkoxy;
R₃ is C₁-C₃ alkoxy; and
X is C₆-C₁₀ alkenyl, substituted with oxo, —OH, and two C₁-C₃ alkoxy groups.

6. The compound as claimed in claim 3, wherein
R₁ is H;
R₂ is C₁-C₃ alkoxy;
R₃ is C₁-C₃ alkoxy; and
X is C₈-C₁₂ alkenyl substituted with oxo and C₁-C₃ alkoxy.

7. The compound as claimed in claim 3, wherein
R₁ is OH;
R₂ is C₁-C₃ alkoxy;
R₃ is C₁-C₃ alkoxy; and
X is C₄-C₈ alkenyl substituted with two oxo groups.

8. The compound as claimed in claim 3, wherein
R₁ is C₁-C₃ alkoxy;
R₂ is OH;
R₃ is C₁-C₃ alkoxy; and
X is C₄-C₈ alkenyl substituted with two oxo groups.

9. The compound as claimed in claim 3, wherein
R₁ is H;
R₂ is OH;
R₃ is OH; and
X is C₁-C₆ alkyl substituted with ═NH.

10. The compound as claimed in claim 3, wherein
R₁ is OH;
R₂ is C₁-C₃ alkoxy;
R₃ is C₁-C₃ alkoxy; and
X is C₂-C₃ alkenyl substituted with OH.

11. The compound as claimed in claim 3, wherein
R₁ is H;
R₂ is C₁-C₃ alkoxy;
R₃ is C₁-C₃ alkoxy; and
X is C₈-C₁₂ alkenyl substituted with oxo and 2 C₁-C₃ alkoxy groups.

12. The compound as claimed in claim 3, wherein
R₁ is OH;
R₂ is C₁-C₃ alkoxy;
R₃ is C₁-C₃ alkoxy; and
X is aralkyl comprising a C₆-C₈ alkyl and a C₆ aryl, wherein the alkyl is substituted with oxo and the aryl is substituted with two C₁-C₃ alkoxy groups.

13. A compound of Formula 2 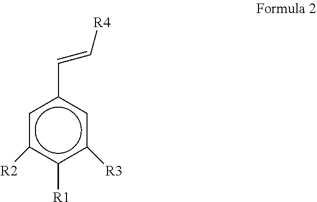
or any derivative thereof, pharmaceutically acceptable salt thereof, or combination thereof wherein:
R₁ is H or OH;
R₂ is alkoxy or OH;
R₃ is alkoxy or OH; and
R₄ is an C₁-C₁₅ alkyl, C₂-C₁₅ alkenyl, or aralkyl, each of which is substituted with at least one alkoxy, —OH, or oxo.

14. The compound as claimed in claim 13, wherein R₄ is selected from the group consisting of:

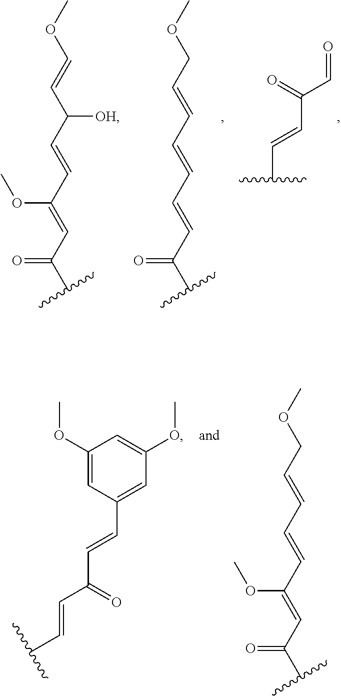

15. The compound as claimed in claim 13, wherein
R₁ is H;
R₂ is OCH₃;
R₃ is OCH₃; and
R₄ is

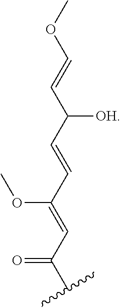 [SMILES: *C(=O)/C=C(/C=C/C(O)/C=C/OC)OC]

16. The compound as claimed in claim 13, wherein
R₁ is H;
R₂ is OCH₃;
R₃ is OCH₃; and
R₄ is.

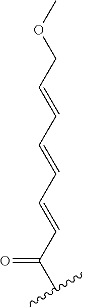 [SMILES: *C(=O)/C=C/C=C/C=C/COC]

17. The compound as claimed in claim 13, wherein
R₁ is OH;
R₂ is OCH₃;
R₃ is OCH₃; and
R₄ is

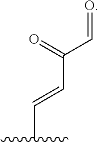 [SMILES: */C=C/C(=O)C=O]

18. The compound as claimed in claim 13, wherein
R₁ is OH;
R₂ is OCH₃;
R₃ is OCH₃; and
R₄ is

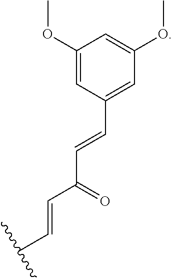 [SMILES: */C=C/C(=O)/C=C/c1cc(OC)cc(OC)c1]

19. The compound as claimed in claim 13, wherein
R₁ is H;
R₂ is OCH₃;
R₃ is OCH₃; and
R₄ is

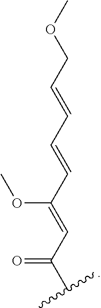

20. A compound of Formula 3 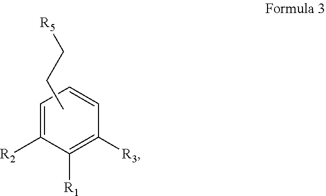
or any derivative thereof, pharmaceutically acceptable salt thereof, or combination thereof wherein:
R₁ is H, alkoxy, or OH;
R₂ is alkoxy or OH;
R₃ is alkoxy or OH; and
R₅ is C₁-C₁₂alkyl or C₂-C₁₂ alkenyl, which is independently substituted with 1, or 2, substituents selected from the group consisting of ═NH and oxo.

21. The compound as claimed in claim 20, wherein
R₁ is H;
R₂ is OH;
R₃ is OH; and
R₅ is ═NH, wherein
R₅ is in the ortho position to R₂.

22. The compound as claimed in claim 20, wherein
R₁ is OC₂H₅;
R₂ is OH;
R₃ is OCH₃; and
R₅ is 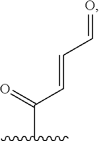
wherein
R₅ is in the para position to R₁.

23. A compound Formula 4; 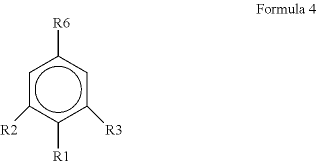
or any derivative thereof, pharmaceutically acceptable salt thereof, or combination thereof wherein:
R₁ is OH;
R₂ is C₁-C₃ alkoxy;
R₃ is C₁-C₃ alkoxy; and
R₆ is C(CH₂)OH.

24. The compound as claimed in claim 23, wherein
R₁ is OH;
R₂ is OCH₃;
R₃ is OCH₃; and
R₆ is C(CH₂)OH.

25. A pharmaceutical composition, comprising a compound of claim 1 and a pharmaceutically acceptable carrier.

26. A method of treating a chronic disorder in a patient in need thereof, comprising administering the compound of claim 1.

27-40. (canceled)
